(19) United States
(12) Patent Application Publication
(10) Pub. No.: US 2014/0336593 A1
(43) Pub. Date: Nov. 13, 2014

(54) DRUG DELIVERY DEVICES AND METHODS OF USE THEREOF

(71) Applicant: ON DEMAND THERAPUTICS, INC., Menlo Park, CA (US)

(72) Inventors: Jonathan R. Coppeta, Windham, NH (US); Robert Dyer, Concord, MA (US); Sheryl Kane, Somerville, MA (US); Vipul Taneja, North Chelmsford, MA (US); John T. Santini, JR., Chanhassen, MN (US); Catherine Santini, Chanhassen, MN (US)

(21) Appl. No.: 14/445,177

(22) Filed: Jul. 29, 2014

Related U.S. Application Data
(63) Continuation of application No. PCT/US2013/025188, filed on Feb. 7, 2013.
(60) Provisional application No. 61/595,984, filed on Feb. 7, 2012.
(60) Provisional application No. 61/640,683, filed on Apr. 30, 2012.
(60) Provisional application No. 61/659,668, filed on Jun. 14, 2012.

(51) Int. Cl. A61F 9/00

(52) U.S. Cl. CPC A61F 9/0017; A61F 2230/0069; A61F 2250/0014; A61F 2250/0071
USPC 604/244

(57) ABSTRACT

Drug delivery devices are provided that are configured to release drug following passive or active activation of the device protecting the drug contained therein. In one aspect, the device may be configured to release a drug following selective application of light irradiation to the device. In another aspect, the device is configured to a release drug following degradation of at least a part of the device body that is formed, for example, from a bioerodible, hermetic material. An exemplary bioerodible, hermetic material is a biodegradable glass. Still other aspects provide for release of a drug upon a combination of both passive and active activation of the device.

### CROSS-REFERENCE TO RELATED APPLICATIONS

This application is a continuation of International Application No. PCT/US2013/25188, filed Feb. 7, 2013, which claims benefit of U.S. Provisional Patent Application No. 61,595,984 filed Feb. 7, 2012; U.S. Provisional Patent Application No. 61/640,683 filed Apr. 30, 2012; and U.S. Provisional Patent Application No. 61/659,668 filed Jun. 14, 2012. These earlier applications are incorporated herein by reference.

### BACKGROUND

The present disclosure is generally directed to medical devices for controlled drug delivery, and, more particularly, is directed to implantable devices and methods for delivering an active pharmaceutical ingredient to a tissue site in a patient's body, such sites including but not limited to the eye for the treatment of ocular diseases and conditions.

Drug-eluting devices that may be implanted directly into the eye generally are known. These devices may be surgically implanted or are injected into the posterior chamber or into, onto, or under layers of the eye such as the conjunctiva, sclera, or choroid. Commercially available drug eluting implants include ganciclovir implants (Vitrasert®) for treatment of CMV retinitis in patients with acquired immunodeficiency syndrome (AIDS), fluocinolone acetonide implants (e.g., Retisert®) for treatment of chronic non-infectious uveitis of the posterior segment of the eye, and dexamethasone implants (e.g., Ozurdex®) for treatment of macular edema caused by retinal vein occlusions and for chronic non-infectious uveitis of the posterior segment of the eye. Notable implants in development include an injectable fluocinolone acetonide implant (Iluvien™) for the treatment of diabetic macular edema (DME) and a bioerodible latanoprost implant (Durasert™) for treatment of glaucoma and ocular hypertension. Typically, such implants release a drug at a constant or slowly changing rate. See, U.S. Pat. Nos. 6,217,895 and 6,548,078 (to Retisert), U.S. Pat. No. 5,378,475 (to Vitrasert), U.S. Pat. Nos. 6,726,918; 6,899,717; 7,033,605; 7,625,582; 7,767,223 (to Ozurdex), and U.S. Patent Application Publication 2007/0122483 (to Iluvien). These implantable devices typically provide a constant pharmacokinetic profile resulting from a continuous drug dosing. This continuous dosing may be acceptable for certain drugs, but for other drugs, continuous dosing can result in serious side effects. For example, continuous delivery of a steroid in the eye results in a high incidence of cataracts or elevated intraocular pressure that may result in glaucoma. Thus, in some cases, it is desirable to deliver the drug only when needed, for example at spaced time intervals.

Another significant challenge in the development of technologies for the delivery of pharmaceutical drugs and, in particular, macromolecule (e.g., peptide and protein) drugs, is the limited stability of these molecules when in contact with water vapor or when in an aqueous solution. Many macromolecule drugs, including proteins, that are unstable in aqueous solution are handled and stored as dry solids (“dry” as defined herein mean substantially free of residual moisture, typically with a water content not exceeding 10% water by weight). Delivery systems that store or release macromolecule drugs in liquid or gel form will have limited utility due to accelerated degradation of the drug caused by high residual moisture. If a macromolecule drug can be kept in a dry, solid form, then its degradation can be minimized and a long-term implantable device is possible. See Proos, et al., “Long-term Stability and In Vitro Release of hPTH(1-34) from a Multi-reservoir Array” Pharmaceutical Research, 25(6): 1387-95 (2008). It is therefore desirable to create a drug delivery system that has the ability to store a drug in a dry, solid form and that prohibits or limits any moisture from passing through the device and into the drug, until such time that release of the drug is desired.

PCT WO 2009/097468 to Kliman discloses drug delivery devices that may be configured for implantation into an ocular region of a subject, where drug release may be triggered by an optical stimulus, such as light having a certain wavelength.

There remains a need, however, for an improved implantable drug delivery device for delivering a drug to the interior of the eye. In particular, a need exists for an implantable drug delivery device having a simple and compact construction that provides a hermetic barrier to protect a sensitive drug payload until the payload is selectively released and that is capable of laser activation so that a drug dosing can be initiated at a selected time using non-invasive techniques. Desirably, such a device should be easy to manufacture and should not require on-board electronics.

### SUMMARY

In one aspect, a drug delivery device is provided that is configured to release drug following the selective application of light irradiation to the device while protecting the drug contained therein. In one example, the device includes a tube element having a reservoir enclosed therein; a drug unit contained in the enclosed reservoir, the drug unit including a drug; and a shielding element contained in the enclosed reservoir, wherein the drug delivery device is configured to absorb light irradiation from a laser source effective to rupture the tube element, thereby opening the enclosed reservoir to permit release of the drug from the drug delivery device, and the shielding element being configured to shield the drug unit from the light irradiation. In another example, the device includes a tube element having a reservoir enclosed therein; and a drug unit contained in the enclosed reservoir, the drug unit including a drug, wherein the drug delivery device is configured to absorb light irradiation from a laser source effective to rupture the tube element, thereby opening the enclosed reservoir to permit release of the drug from the drug delivery device, and wherein the drug unit is shaped and dimensioned to reside in the enclosed reservoir at a position which creates a buffer zone between a portion of an inner wall of the tube element and the drug unit, whereby the buffer zone reduces or eliminates exposure of the drug unit to the light irradiation or heat therefrom. In embodiments, the reservoirs are hermetically sealed. The devices may be configured for implantation in a patient for release of one or more doses of drug over an extended period.

In another aspect, a drug delivery device is provided that includes a device body having at least one enclosed reservoir therein; a bioerodible, hermetic material defining at least a portion of the at least one enclosed reservoir; and a drug unit disposed in the at least one enclosed reservoir, the drug unit including a drug; wherein the bioerodible, hermetic material includes a biodegradable glass configured to degrade when contacted with a biological fluid, thereby to open the enclosed reservoir and permit release of the drug therefrom. In embodiments, the reservoirs are hermetically sealed. The devices may be configured for implantation in a patient for release of one or more doses of drug over an extended period. The device may be partially or completely biodegradable.

In still another aspect, a method is provided for releasing at least two separate doses of a drug from a drug delivery device. In one embodiment, the method includes: (i) deploying the drug delivery device into an aqueous fluid, the drug delivery device having an elongated tubular housing which comprises at least two hermetically sealed reservoirs therein, each of the reservoirs containing a dose of the drug in a dry solid form; (ii) directing light irradiation from a laser energy source to an exterior surface of drug delivery device to rupture a first of the hermetically sealed reservoirs, thereby permitting at a first time ingress of the aqueous fluid into the first reservoir, dissolution of the dose of the drug in the first reservoir, and release of the dissolved dose of drug from the first reservoir and out of the drug delivery device; and subsequently (iii) permitting the aqueous fluid in the first reservoir to contact and biodegrade a hermetic barrier element separating the first reservoir and a second hermetically sealed reservoir, thereby permitting at a second and later time ingress of the aqueous fluid into the second reservoir, dissolution of the dose of the drug in the second reservoir, and release of the dissolved dose of drug from the second reservoir, through the first reservoir, and out of the device.

In various embodiments, the devices of the first and second aspects mentioned above may be combined together, and the method of the third aspect mentioned above may be carried out using the devices of the first and/or second aspects mentioned above.

### BRIEF DESCRIPTION OF THE DRAWINGS

FIGS. 1A-1C are an exploded view (A), a cross-sectional view (B), and a perspective view (C) of an embodiment of an implantable drug delivery device.

FIGS. 2A and 2B are exploded views of two embodiments of an implantable drug delivery device having a v-shaped shielding element (A) and a sphere-shaped shielding element (B).

FIGS. 3A-3C are cross-sectional views of three embodiments of an implantable drug delivery device having a cylindrical band shielding element (A), a cylindrical coil shielding element (B), and a perforated element shielding element (C).

FIG. 4 is a cross-sectional view of an embodiment of an implantable drug delivery device in which a portion of the drug unit is shaped to provide a buffer area.

FIG. 5 is a perspective view of an embodiment of an implantable drug delivery device having one or more biodegradable structural elements.

FIGS. 6A-6D are plan and cross-sectional views of four exemplary embodiments of end cap elements.

FIG. 7 is a schematic illustration of a method for forming a glass tube element having a sealed end for use in an implantable drug delivery device.

FIG. 8 is a schematic illustration of a method for forming a glass tube element for use in an implantable drug delivery device.

FIG. 9 is a schematic illustration of a method for forming an integral end cap element on a glass tube element for use in an implantable drug delivery device.

FIG. 10 shows plan views of two embodiments of a glass tube element having an end cap element for use.

FIG. 11 is a perspective view of an implantable drug delivery device having separate reservoir components.

FIGS. 12A-12G are cross-sectional views of one embodiment of the operation of the implantable drug delivery device illustrated in FIG. 11.

FIGS. 13A-13C are cross-sectional views of another embodiment of the operation of the implantable drug delivery device illustrated in FIG. 11. FIG. 13D is a graphical illustration of the drug release according to method of operation illustrated in FIGS. 13A-13C.

FIG. 14 shows cross-sectional views of one embodiment of an implantable drug delivery device and its operation.

FIGS. 15A-15D are perspective views of embodiments of configurations for joining separate reservoir sections in implantable drug delivery devices.

FIG. 16 is a cross-sectional view of an embodiment of an implantable drug delivery device having several separate reservoir sections.

### DETAILED DESCRIPTION

The embodiments described herein relate to drug delivery devices (DDDs) such as implantable DDDs that provide one or more hermetically-sealed reservoirs that are capable of providing a controlled release of one or more doses of an active pharmaceutical ingredient. In one aspect, the DDDs are capable of being implanted into a tissue of the eye and subsequently activated by an ocular laser to permit the drug to be released in ocular tissues without damaging the drug. In another aspect, the release of the drug from the DDDs is passively controlled following biodegradation or bioerosion of a material defining at least a portion of the reservoir. In still another aspect, the release of multiple doses of drug from a single device utilizes a combination of activation by an ocular laser and biodegradation or bioerosion of a material defining the reservoirs.

The devices and methods described herein allow for the selective release of a drug by laser irradiation of a DDD having a hermetically-sealed reservoir or reservoirs, biodegradation or bioerosion of a material defining a hermetically-sealed reservoir or reservoirs, or combinations thereof. The devices described herein are formed of materials and arranged in structures that provide the drug in a hermetic reservoir capable of either being breached by laser irradiation or biodegradation or bioerosion of the materials.

Certain constraints exist in forming an implantable DDD having a hermetically-sealed reservoir for containing and releasing a drug. For example, to facilitate implantation, the DDD should have a dimension sufficiently small so as to allow injection into or implantation into a target tissue site. In one aspect, the target tissue site is in the ocular tissue. In another aspect, the target tissue site is in the brain tissue. The DDD also should be sufficiently rigid to withstand implantation while maintaining the hermetic seal over the one or more reservoirs. Additionally, structural joints of the DDD should be robustly formed to prevent compromise of the reservoir during implantation and while the DDD is implanted in the tissue prior to activation. Furthermore, the DDD should have a simple construction that is easy for manufacture from biocompatible materials and assemble for use.

Other constraints exist in forming an implantable DDD that is capable of providing controlled release of a drug. For example, at least a portion of implantable DDDs configured for laser-activated release of a drug should be able to absorb light irradiation from a laser source effective to open one or more hermetically-sealed reservoirs to permit the drug to be released. The irradiation-absorbing portion should be large enough to be specifically targeted by an ocular laser. Additionally, the irradiation-absorbing portion should be formed of a biocompatible material that is capable of being breached upon exposure to a minimal amount of laser energy. Furthermore, the irradiation-absorbing portion should have a thickness that is capable of being breached upon exposure to a minimal amount of laser energy.

Still other constraints exist in use of implantable DDDs configured for laser-activated release of a drug. For example, implantable DDDs configured for laser-activated release of a drug can require multiple laser activations of the hermetically-sealed reservoirs to release the drug from multiple reservoirs. Thus, it is desirable for the implantable DDDs to be configured to provide controlled release of the drugs using a single activation stimulus or less frequent application of an activation stimulus.

The implantable DDDs described herein have been developed to provide a desirable balance in addressing these competing constraints. The elements of the DDD are relatively easy to manufacture from biocompatible metals and glasses, and assembly is relatively straightforward due to the simple features of construction.

For example, according to one embodiment, the implantable DDD includes a glass tube element, metal end cap elements, and a metal coating, which together provide an appropriate reservoir for containing and releasing a drug. The reservoir is formed by the glass tube element and the metal end cap elements joined to opposing ends of the glass tube element. The metal end cap elements may be joined to the glass tube element by an adhesive, ensuring that the metal end cap elements are securely bonded to the glass tube. The metal coating forms a seal over the joints, ensuring that the reservoir is hermetically sealed to maintain the integrity of the drug. The metal coating also may cover some or all of the glass tube element to provide a target area capable of absorbing light irradiation from a laser source effective to open the hermetically-sealed reservoir to permit the drug to be released. In embodiments, the metal coating and the glass tube element are sufficiently rigid to withstand implantation, yet they are able to be breached upon exposure to a minimal amount of laser energy. In other embodiments, the metal coating and the glass tube element are sufficiently rigid to withstand implantation, yet the glass tube element is able to be breached upon biodegradation or bioerosion of the glass tube element.

The DDD may further comprise a shielding element disposed in at least a portion of the reservoir. The shielding element advantageously protects the drug from being inadvertently damaged by application of the activation stimulus. According to embodiments, the DDD may further comprise multiple barrier elements positioned within the enclosed reservoir, the barrier elements defining a plurality of separate reservoir sections that can be used to provide a variety of drug release profiles. Desirably, the multiple barrier elements comprise a hermetic material to hermetically seal each separate reservoir section from adjacent separate reservoir sections.

The hermetically-sealed reservoirs of the implantable DDDs described herein beneficially enable the use of sensitive drugs in an implant device intended for deployment in a patient over an extended period. For example, some treatment regimens require sustained or multiple releases of a drug over a period ranging from a week to several months, a year, or more. For drugs that are sensitive to water or air exposure, a hermetically-sealed reservoir protects the drug payload and eliminates or minimizes drug degradation over the extended period.

In embodiments, the implantable DDDs described herein are configured for the non-invasive release of a drug to the tissue being treated, such as the macula or retina, by releasing the drug into the posterior chamber through the vitreous portion of the eye or through the conjunctiva, sclera, or choroid. In addition to being non-invasive, laser activation or passive activation allows for release of multiple, discrete doses of drug from a single implanted device. Multiple dosing allows the dosing interval to be tailored, providing some control over the drug concentration over time. Laser activation also advantageously allows a physician to precisely control the initiation of treatment and administer arbitrary and customized treatment regimens. The selectable nature of the activation and dosing is not realized in existing passive drug delivery implant devices.

The individual reservoirs or reservoir sections of the implantable DDDs described herein are independent of the drug formulation and allow the integration of different drug forms and types in the overall device. By encapsulating a different drug in each reservoir or reservoir section, an optimal formulation for each drug can be developed. The overall device therefore can enable multiple drug therapies within one implantable device. In addition, when appropriate, multiple drugs can be co-formulated within one reservoir or reservoir section.

### 1. Laser-Activated Drug Delivery Devices

In one aspect, embodiments described herein include DDDs that can be implanted into an ocular tissue with minimal intervention, are hermetically sealed to protect a drug payload over time, and can be laser activated to selectively initiate release of one or more doses of the drug, as needed. The primary components of certain embodiments of the DDD described herein include: structural elements forming an enclosed reservoir, and at least one drug unit contained in the enclosed reservoir. The drug unit includes at least one drug. The enclosed reservoir is hermetically sealed by the structural elements and/or a coating to maintain the biologic activity or chemical viability of the drug until the reservoir is intentionally breached (i.e., by application of laser energy) to permit the drug to be released to one or more target tissues at or around the site of implantation. For example, in some embodiments, one or more of the structural elements are formed of a hermetic material, preventing air and water from entering the enclosed reservoir. In some embodiments, one or more of the structural elements are formed of a hermetic material, preventing air and water from entering the enclosed reservoir. In some embodiments, the coating may form a hermetic seal over joints of the structural elements to prevent air and water from entering the enclosed reservoir. In some embodiments, one or more of the structural elements is formed of a non-hermetic material, and the coating forms a hermetic seal over such elements to prevent air and water from entering the enclosed reservoir. In preferred embodiments, the DDD has an elongated, cylindrical shape and has a small enough outer diameter to permit in vivo insertion of the DD using a narrow diameter applicator, such as a syringe needle.

The materials and construction of the implantable DDDs described herein account for the competing constraints with respect to hermeticity, laser activation, implantability, and manufacturability of the devices. Some polymers are well suited to thin-walled construction and are compatible with laser activation. For example, some polymers may be easily manufactured as thin-walled elements and may be breached using a pulse of laser radiation. However, these polymers may not provide sufficiently low water vapor barrier characteristics required for some of the drugs of interest. Metals, glasses, and ceramics, on the other hand, offer superior barrier characteristics. These materials generally require higher laser energy to breach; however, the DDDs described herein use these materials in the form of thin walls and/or coatings, so that they can be breached with a minimal amount of laser energy.

In embodiments, the thin walls that form the enclosed reservoir are relatively thin (e.g., 1 μm to 100 μm), depending on the particular laser mechanism employed (thermal, thermo-mechanical, photo-chemical, photo-disruptive, etc.). In embodiments, the wall thickness ranges from 1 μm to 75 μm, preferably from 5 μm to 50 μm, and more preferably from 5 μm to 15 μm. If the DDD is to be injected into an ocular cavity, the enclosed reservoirs will typically have an internal cavity diameter ranging from 50 μm to 500 μm. Depending on the length of the DDD, the volume of the enclosed reservoir or reservoir section typically ranges from 0.1 μL to 10 μL. Larger values may be used depending on the method and site of implantation. The dimension range may be higher for non-ocular applications.

Although the reduced amount of laser energy helps reduce inadvertent damage to the drug disposed in the reservoir that may be caused by application of the laser energy, embodiments of the DDDs described herein may further include a shielding element in the enclosed reservoir to provide a buffer, or shield, between the drug and the laser energy. The shielding element may be configured in any suitable shape or size to fit within the enclosed cavity with the drug unit and may be made from any suitable material. For example, in embodiments the shielding element includes a three-dimensional structure disposed in the enclosed reservoir adjacent to the drug unit.

The drug units of the DDDs contain one or more drugs. The drug unit may contain one or more excipients. The drug unit may be in the form of an elongated tablet or a capsule. In a preferred embodiment, the drug unit is a microtablet formulated and made as described in U.S. Pat. No. 8,192,659 to Coppeta, et al., which is incorporated herein by reference.

The DDDs described herein can be used with essentially any drug, or active pharmaceutical ingredient (API). In a preferred embodiment, the drug is selected from potent biomolecules, such as proteins, antibodies, vaccines, RNA, DNA or the like. In other embodiments, the drug is selected from small molecule pharmaceuticals. In one embodiment, the drug is an anti-VEGF drug. Examples of such drugs include the antibody fragment ranibizumab/Lucentis™, the antibody bevacizumab/Abastin™, and the fusion protein aflibercept/Eylea™. In other embodiments, the drug may be selected from the group consisting of anti-angiogensis agents, anti-inflammatories, anti-infectives, anti-allergens, cholinergic agonists and antagonists, adrenergic agonists and antagonists, anti-glaucoma agents, agents for cataract prevention or treatment, neuroprotection agents, anti-oxidants, antihistamines, anti-platelet agents, anti-coagulants, anti-thrombic agents, anti-scarring agents, anti-proliferatives, anti-tumor agents, complement inhibitors, decongestants, vitamins, growth factors, anti-growth factor agents, gene therapy vectors, chemotherapy agents, protein kinase inhibitors, small interfering RNAs, antibodies, antibody fragments, fusion proteins, limus family compounds, and combinations thereof. Examples of suitable excipients include but are not limited to lyoprotectants, binding agents, buffers, surfactants, and/or slip agents, all of which are known in the art.

The following exemplary embodiments of implantable DDDs provide one or more hermetically sealed reservoirs that are capable of providing a laser-activated release of a drug.

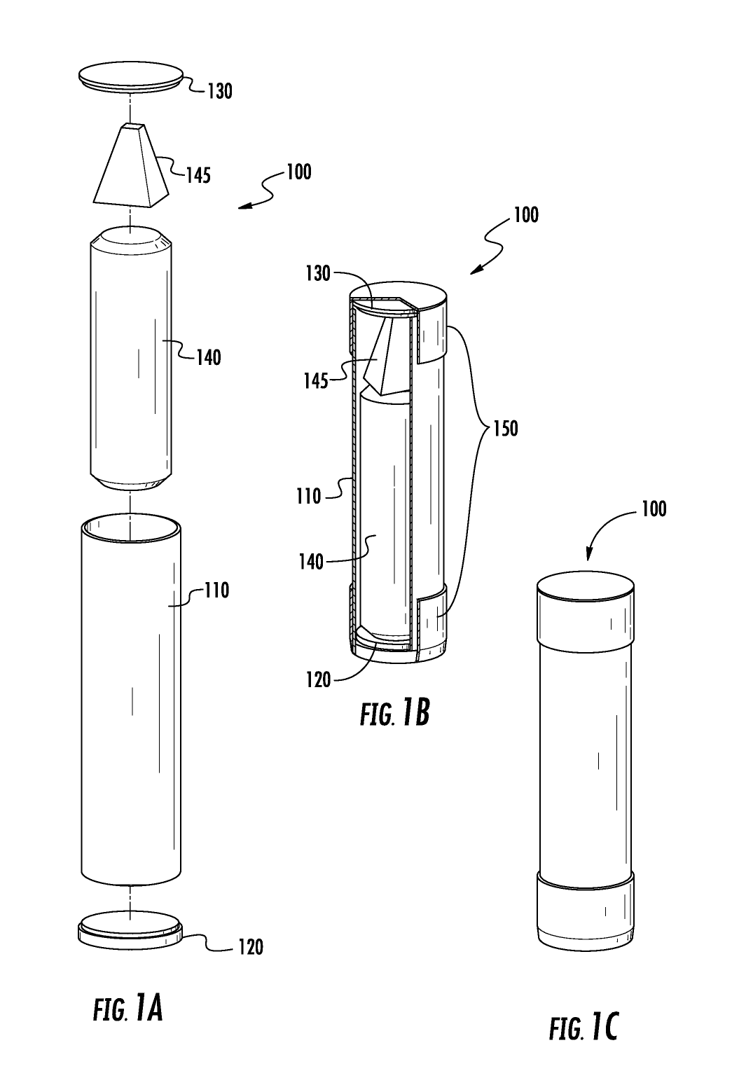

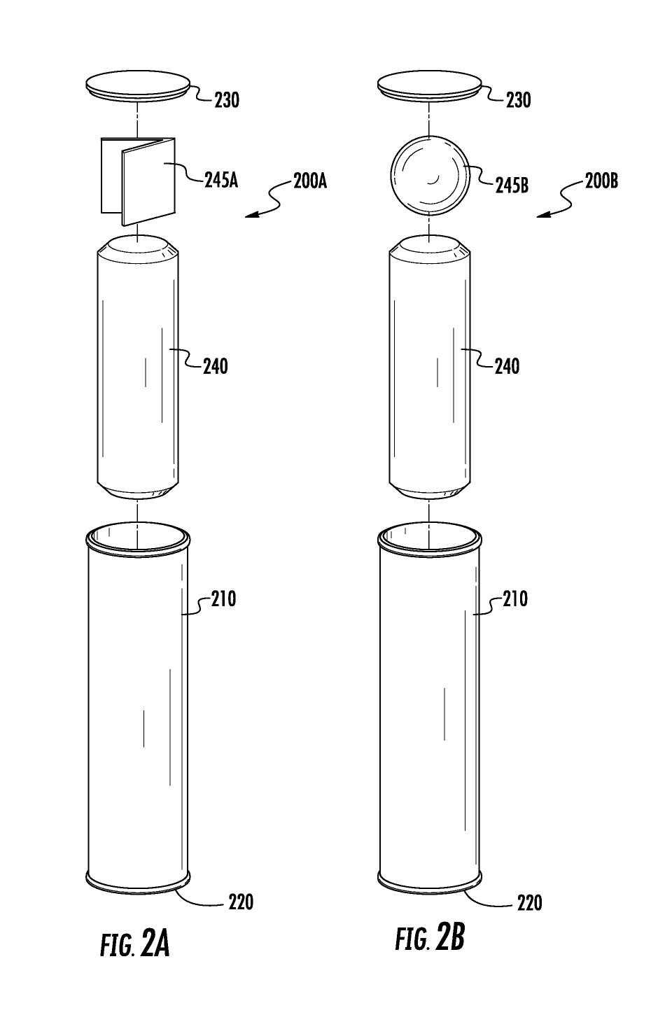

FIGS. 1A and 1B show an embodiment of an implantable DDD 100 including a tube element 110. Before device assembly is completed, the tube element 110 has a first open end and a second open end. In some embodiments, the tube element 110 is formed of a hermetic material, providing a highly impermeable barrier to air and water. For example, in embodiments the tube element is formed of a glass. The glass may be relatively brittle or fragile, and thus can be breached easily during laser activation of the DDD 100. Non-limiting examples of suitable glasses for the tube element 110 are semi-crystalline quartz, photo-lithographically constructed semi-conductor structures, fused silica, and Apex photodefinable glass. In one embodiment, a thin-walled microcapillary glass tube is used as the tube element 110. A commercially available example is part number TSP320450 produced by PolyMicro, Inc. This tube is a fused silica capillary of outer diameter 450 μm and inner diameter 320 μm, which corresponds to a glass wall thickness of 65 μm. Another example is part number BG-05 produced by Charles Supper Co., which has an outer diameter of 500 μm and a wall thickness of 10 to 15 μm Thinner walls are desirable, such as walls having a thickness of 5 to 10 μm, but fabrication techniques may limit achievable wall thicknesses of microcapillary glass tubes. In one embodiment the glass tube element 110 is able to absorb light irradiation from a laser source effective to breach the tube element 110. For example, the glass tube element 110 may be formed with an absorber. A commercially available example of a glass formed with an absorber is RG1000 visible light absorbing glass produced by Schott Glass.

In some embodiments, the tube element 110 is formed of a ductile metal providing a highly impermeable barrier to air and water. Non-limiting examples of suitable metals for the tube element 110 are titanium and gold. In one embodiment, the tube element 110 is produced using conventional extrusion techniques or using a co-extrusion technique for ultra-thin walls (e.g., 5 to 10 μm). According to co-extrusion techniques, the core of a wire can be made of a selectively etchable material to create a hollow tube structure after etching. A commercially-available example of a co-extruded wire is produced by Anomet for the medical industry. In one embodiment, the metal tube element 110 is able to absorb the light irradiation from a laser source effective to breach the tube element 110.

In other embodiments, the tube element 110 is formed of materials other than glass or metal, which materials are highly impermeable to air and water and are able to be breached easily during laser activation of the DDD 100.

The implantable DDD 100 also includes a first end cap element 120 and a second end cap element 130. The first end cap element 120 is joined to the first open end of the tube element 110 at a first joint, and the second end cap element 130 is joined to the second open end of the tube element 110 at a second joint. Accordingly, the tube element 110, the first end cap element 120, and the second end cap element 130 form an enclosed reservoir. In some embodiments, the end cap elements 120, 130 are formed of a metal providing a highly impermeable barrier to air and water. Non-limiting examples of suitable metals for the end cap elements 120, 130 are titanium and gold. In some embodiments, the end cap elements 120, 130 are formed of a glass, silicon, or other ceramic material. In one embodiment, as shown in FIG. 1A, the end cap elements 120, 130 each include a smaller diameter portion and a larger diameter portion. Accordingly, the end cap elements 120, 130 have a T-shaped cross-section and an axially symmetric shape. The smaller diameter portion is inserted into an open end of the tube element 110, and the larger diameter portion contacts an end edge of the tube element 110, forming a joint. Accordingly, the end cap elements 120, 130 are partially received in the tube element 110 at the first joint and the second joint, respectively. In one embodiment, the end cap elements 120, 130 are formed of disks having a constant diameter, and the end cap elements 120, 130 are entirely received in the tube element 110 at the first joint and the second joint, respectively. In one embodiment, the end cap elements 120, 130 are formed as metal foils that are ultrasonically bonded to the open ends of the tube element 110.

In some embodiments, the integrity of the joints between the end cap elements 120, 130 and the tube element 110 is enhanced by use of an adhesive applied to the end cap elements 120, 130, the tube element 110, or both. For example, the adhesive may be a polymer, non-limiting examples of which include an epoxy, a thermoplastic polymer, a thermoset polymer, and other polymeric materials commonly used to create an adherent layer for bonding or sealing. In one embodiment, the adhesive is a pre-coating material. Non-limiting examples of suitable pre-coating materials are gold, titanium, platinum, and other pre-coating materials commonly used to create an adherent layer for bonding or sealing. In one embodiment, the adhesive is applied only to interfacing surfaces of the end cap elements 120, 130 and the tube element 110. In one embodiment, the adhesive is applied only to non-interfacing surfaces of the end cap elements 120, 130 and the tube element 110. Use of the adhesive is advantageous when the end cap elements 120, 130 and the tube element 110 are formed of dissimilar materials. For example, use of the adhesive is particularly advantageous when the end cap elements 120, 130 are formed of a metal and the tube element 110 is formed of a glass because the adhesive serves to bond and seal the joints of the dissimilar materials. Glass-metal seals of this type can be used in high vacuum applications in which pressures as low as 10⁻¹⁰ Torr are maintained, making this seal type an excellent choice for creating a hermetic seal between glass and metal elements.

In some embodiments, the end cap elements 120, 130 are joined to the tube element 110 by welding or soldering the end cap elements 120, 130 to the tube element 110. The use of welding or soldering to bond the elements is particularly advantageous when the end cap elements 120, 130 and the tube element 110 are formed of a metal because the welding or soldering forms a hermetic seal over the first joint and the second joint, respectively. Non-limiting examples of welding or soldering techniques include ultrasonic welding, compression welding, resistive welding, cold-welding, and low-temperature soldering. In one embodiment, the end cap elements 120, 130 are coated with a metal that is amenable to welding, such as gold, titanium, or stainless steel, and the end cap elements 120, 130 are welded accordingly to the tube element 110. The coating material may be electroplated or vapor deposited onto the end cap elements 120, 130 to achieve the desired thickness.

The implantable DDD 100 further includes at least one drug unit 140 contained in the enclosed reservoir formed by the tube element 110 and the end cap elements 120, 130. The drug unit 140 generally is inserted into the tube element 110 with one of the end cap elements 120, 130 already joined to the tube element 110. The other of the end cap elements 120, 130 is then joined to the tube element 110, enclosing the reservoir around the at least one drug unit 140. The drug unit 140 includes at least one drug.

In some embodiments, the implantable DDD 100 further includes at least one shielding element 145. The shielding element 145 is configured to protect the drug from being inadvertently damaged by application of the laser energy to the DDD. It is generally positioned within the reservoir to be interposed between the intended breach point (e.g., the laser target) in the wall of the tube element and the drug unit. For example, in some embodiments the shielding element 145 is disposed in the enclosed reservoir adjacent the drug unit 140 to define a portion of the enclosed reservoir that is devoid of the drug unit 140. In such embodiments, the shielding element 145 may be any suitable size and shape to fit within the enclosed cavity without impeding the desired release kinetics of the drug from the reservoir. For example, as shown in FIG. 1, the shielding element 145 may be a pyramid. In other embodiments of the device 200A, B, shown in FIGS. 2A and 2B, the shielding element is a v-shaped solid (245A) or a sphere (245B) disposed adjacent to a drug unit 240 in a reservoir defined by a tube element 210 and the end cap elements 220, 230. Non-limiting examples of other shapes suitable for use as the shielding element include cones, cylinders, and rectangular solids. Advantageously, the shielding element 145 defines a portion of the DDD 100 to which the laser energy can be directly applied to fracture, perforate, damage or otherwise cause the integrity of the tube element 110 to fail, while protecting the drug unit 140 from thermal degradation or other undesirable side-effects resulting from application of the laser energy.

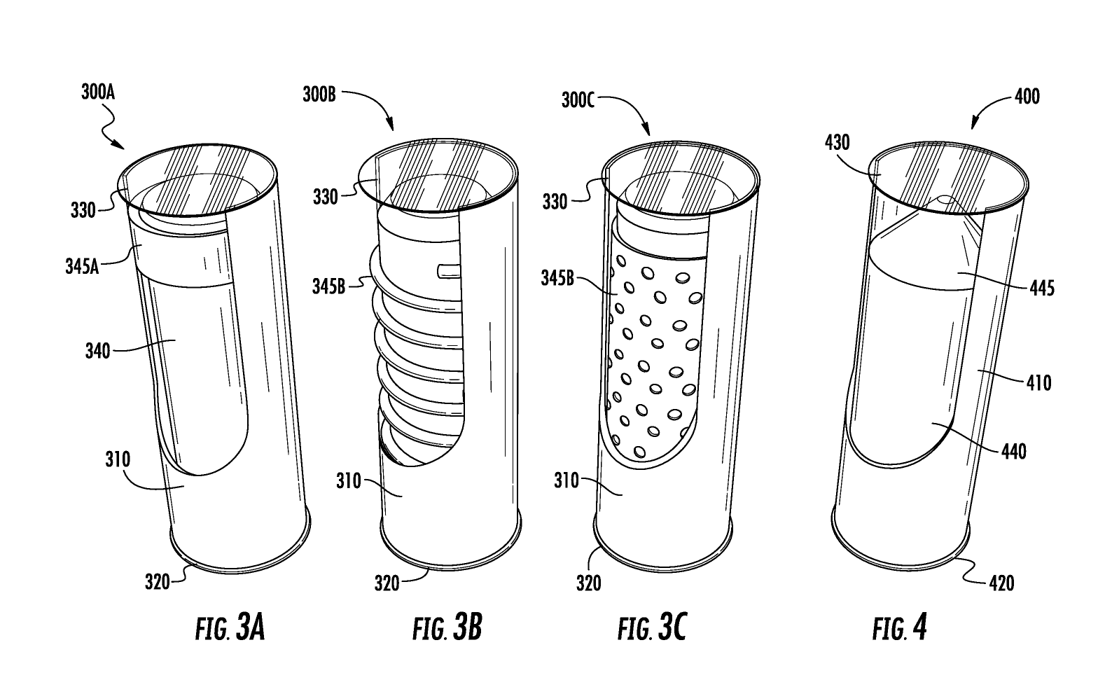

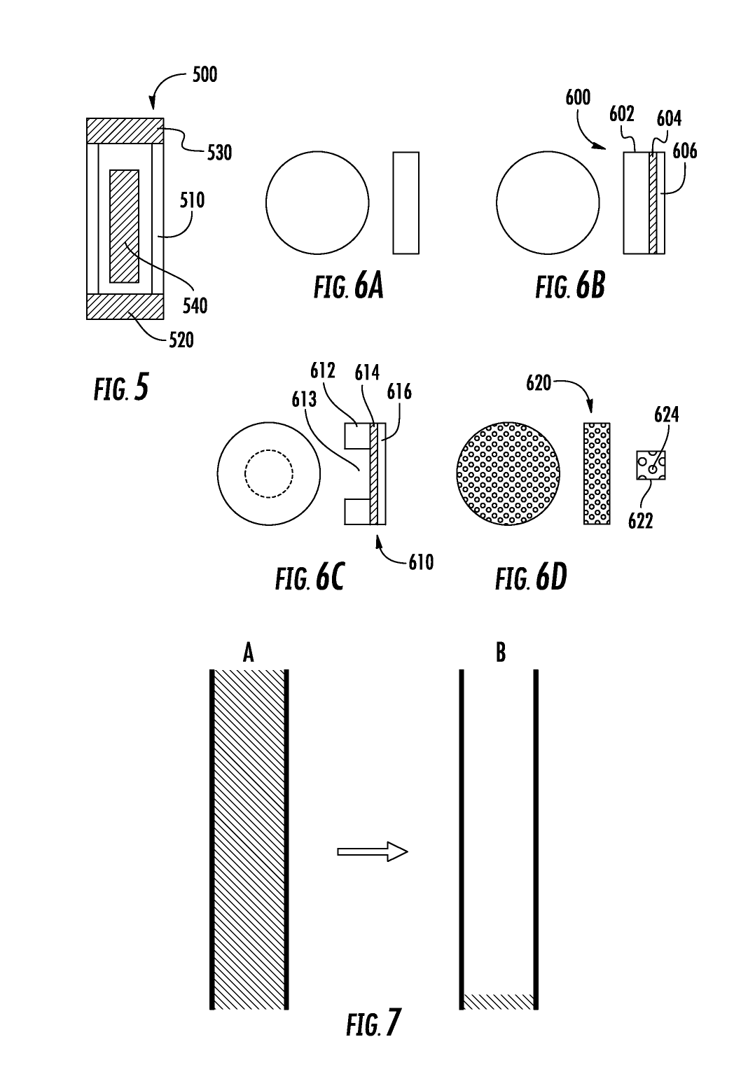

In another embodiment (FIG. 3A-3C), the shielding element 345 is cylindrical structure disposed between at least a portion of the drug unit 340 and an inner wall of the tube element 310 in the DDD 300A, B, C. In such embodiments, the shielding element 345 forms at least a partial barrier between the drug unit 340 and the tube element 310. Non-limiting examples of cylindrical structures that may be used as a shielding element 345 include a cylindrical band 345A, a cylindrical coil 345B, or a perforated cylinder 345C that is disposed around at least a portion of the drug unit in the enclosed reservoir. In another embodiment, the shielding element 345 is a cylindrical semi-permeable membrane having nano-pores or micro-pores disposed around at least a portion of the drug unit 340.

In still another embodiment, the shielding element is an integrally formed from the drug unit, such that a portion of the drug unit is sized and shaped to create a buffer between that portion of the drug unit and the tube element. For example, as shown in FIG. 4, a drug unit 440 may have a first portion and a second portion, the second portion 445 being shaped to provide a buffer between the drug unit 440, the tube element 410, and the end cap elements 420, 430 of the DDD 400. For example, the second portion of the drug unit may be tapered relative to the first portion of the drug unit and have a shape similar to a cone.

In embodiments, the implantable DDD is modified to provide a single tube element having multiple reservoir sections. For example, an embodiment of an implantable DDD may include a tube element, a first end cap element, a second end cap element, and a coating. The tube element, the first end cap element, and the second end cap element form an enclosed reservoir. The DDD also includes at least one barrier element positioned within the enclosed reservoir and defining a plurality of separate reservoir sections. The DDD further includes a plurality of drug units distributed within one or more of the separate reservoir sections. In one embodiment, the at least one barrier element includes a plurality of barrier elements positioned within the enclosed reservoir and defining a plurality of separate reservoir sections. In one embodiment, the plurality of drug units is distributed such that each of the separate reservoir sections contains one drug unit. In one embodiment, the plurality of drug units is distributed such that one or more of the separate reservoir sections contains multiple drug units. In one embodiment, the at least one barrier element and the plurality of drug units are arranged such that multiple drug doses may be released sequentially (and in spaced intervals) from the DDD using a single laser activation event. This single-activation-multiple-releases embodiment is highly advantageous from a patient and physician perspective. A more detailed description of the embodiments of multiple-reservoir DDDs is provided below.

In embodiments, the implantable DDD 100 also includes a coating 150 over all or a portion of the tube element 110 and the end cap elements 120, 130. In one embodiment, the coating 150 is formed of a metal providing a highly impermeable barrier to air and water. Non-limiting examples of suitable metals for the coating 150 include titanium and gold. In one embodiment, the coating 150 is formed of a glass, ceramic, metal alloy, metal laminate, or other hermetic material. In one embodiment, the coating is less than 10 μm thick. In an exemplary embodiment, the coating 150 is formed of a titanium layer that is between 0.2 and 1 μm thick. Methods for making a coating on the implantable DDD may include physical deposition or other coating techniques that produce a contiguous, highly impermeable and inert layer that is thermally coupled to the tube element 110 and the end cap elements 120, 130. Non-limiting examples of physical deposition techniques include sputtering, e-beam evaporative coating, plasma enhanced chemical vapor deposition, atomic layer deposition, and plasma enhanced chemical vapor deposition. In one embodiment, the coating 150 is formed over the joints of the end cap elements 120, 130 and the tube element 110. In one embodiment, the coating 150 is formed over all of the end cap elements 120, 130. For example, when the end cap elements 120, 130 are formed of a non-hermetic material, the coating 150 may be formed over all of the end cap elements 120, 130 to provide a hermetic seal over the end cap elements 120, 130. In one embodiment, the coating is formed over all of the tube element 110 and end cap elements 120, 130. For example, when the tube element 110 and the end cap elements 120, 130 are formed of non-hermetic materials, the coating 150 may be formed over all of the tube element 110 and the end cap elements 120, 130 to provide a hermetic seal over the tube element 110 and the end cap elements 120, 130. In embodiments, the coating 150 is able to absorb light irradiation. For example, when the tube element 110 is formed of a non-irradiation absorbing material, the coating 150 may be formed over all or a portion of the tube element 110 to absorb the light irradiation effective to breach the tube element 110 to permit release of the drug. Conversely, when the tube element 110 is formed of a material that is able to absorb light irradiation, the coating 150 may be formed of a non-irradiation absorbing material over a portion of a tube element 110 to protect certain portions of the tube element 110 from being exposed to the light irradiation (i.e., the coated portions of the tube element) and/or to identify certain portions of the tube element 110 that are desired to be targeted by light irradiation (i.e., the uncoated portions of the tube element).

### 2. Biodegradable Drug Delivery Devices

Although biodegradable materials are described above as being suitable for use as a coating or barrier element, the structural element defining the enclosed reservoir also may be formed from a biodegradable material, so long as the biodegradable material is hermetic. For example, the implantable DDD may have a device body formed at least in part by a wall of a bioerodible or biodegradable hermetic material. The device body defines at least one enclosed reservoir therein. In one embodiment, the implantable DDD is configured to expose the bioerodible or biodegradable material to a biological fluid following laser activation of a coating and/or other element configured to protect the bioerodible or biodegradable material from prematurely opening the enclosed reservoir. In other embodiments, no laser activation is required and enclosed reservoir rupture occurs without intervention, such as by the predetermined bioerosion of the material forming the device body. The hermetic biodegradable materially advantageously can exclude water/humidity from the drug reservoir, keeping the drug dry, stable, and hermetically sealed for an extended period, and yet can control the time of drug release in a manner similar to that of conventional, non-hermetic biodegradable polymers.

As used herein, the terms “bioerodible” and “bioerosion” are used broadly to include dissolution, degradation, erosion, biodegradation by enzymatic action, and the like. The terms “bioerodible” and “biodegradable” are used interchangeably herein unless a particular mechanism is specified.

In a preferred embodiment, the bioerodible hermetic material is a biodegradable glass. As used herein, “biodegradable glass” means any glass formulation that degrades or dissolves in water or other aqueous solutions, including body fluids such as the vitreous or aqueous humors of the eye, subcutaneous fluid, brain/spinal fluid, blood, urine, saliva, or gastric fluid. The network-forming component of the biodegradable glass may be silica, phosphate, borate, or any combination thereof. The biodegradable glass formulation also may include one or more glass modifiers and/or divalent cations. For example, one or more glass modifiers may be included to disrupt or modify the glass forming constituent, non-limiting examples of which include alkali and alkaline earth oxides (e.g., Na₂O, CaO, and MgO). The biodegradable glass formulation also may contain at least one divalent cation, including but not limited to sodium, calcium, magnesium, and potassium, in any ratio. Divalent cations from metal oxides can act as chelating agents between non-bridging oxygen atoms of polymer sections increasing the strength and durability of the glass.

Glass dissolution of the biodegradable glass occurs in two stages: water hydration of a thin glass layer with ion exchange between the hydrated layer and the biological fluid followed by hydrolysis of the network-forming oxygen atoms to create soluble species. The glass dissolution characteristics are governed by the dissolution environment as well as the glass constituents. For example, increasing the divalent glass modifier molar ratio can increase the glass strength and decrease the glass dissolution rate. With respect to the dissolution environment, acidic environments can accelerate glass dissolution by increasing ion exchange and hydrolysis.

Thus, there are a number of parameters to consider in designing an implantable DDD using a biodegradable glass for one or more elements, including dissolution rate, glass formability, glass mechanical strength, biocompatibility, and degradation by-products. The dissolution rate can be controlled by both the glass composition and by geometric considerations. For instance, for biodegradable glasses with an equivalent dissolution rate, a thicker structure will increase the duration the element remains intact. Likewise, the ratio of the exposed surface area of the biodegradable glass to its surface volume can be used to control the duration the element remains intact; a smaller ratio of the exposed surface area to volume may extend the duration. Glass mechanical strength and formability are influenced by the type and ratio of constituents. Parameters such as the glass design strength or the softening temperature compared to the vitrification temperature also may be of importance and will be influenced by the glass constituents. Finally, it generally is important to control the dissolution by-products and any associated interactions with the drug. By-products that form insoluble precipitates or that negatively interact with the drug are undesirable. Non-limiting examples of commercially available biodegradable glass materials include CorGlaes™, produced by GilTech, and some glasses made by MO-SCI Corporation.

The biodegradable glass can be incorporated into embodiments of implantable DDDs having a variety of different configurations. In embodiments, the primary components of the implantable DDDs include: structural elements forming an enclosed reservoir, and at least one drug unit contained in the enclosed reservoir. The drug unit includes at least one drug. The enclosed reservoir is hermetically sealed by the structural elements, and optionally by a coating, to maintain the biologic activity or chemical viability of the drug until the enclosed reservoir is breached either intentionally (e.g., by application of laser energy) or passively (e.g., by dissolution of the biodegradable glass) to permit the drug to be released to one or more target tissues at or around the site of DDD implantation. For example, in some embodiments, one or more of the structural elements of the DDD are formed of the biodegradable glass, preventing air and water from entering the enclosed reservoir. In some embodiments, a coating may form a hermetic seal over joints of the structural elements to prevent air and water from entering the enclosed reservoir. In some embodiments, one or more of the structural elements is formed of a non-hermetic material, and a coating forms a hermetic seal over such elements to prevent air and water from entering the enclosed reservoir. In preferred embodiments, the DDD has an elongated, cylindrical shape and has a small enough outer diameter to permit in vivo insertion of the DDD using a narrow diameter applicator, such as a syringe needle.

A non-limiting example of a DDD 500 is shown in FIG. 5. The DDD 500 includes a tube element 510, which prior to assembly has a first open end and a second open end. The implantable DDD 500 also includes a first end cap element 520 and a second end cap element 530. The first end cap element 520 is joined to the first open end of the tube element 510 at a first joint, and the second end cap element 530 is joined to the second open end of the tube element 510 at a second joint. Accordingly, the tube element 510, the first end cap element 520, and the second end cap element 530 form an enclosed reservoir in which a drug unit 540 is disposed. The drug unit 540 includes at least one drug. At least one of the tube element 510, the first end cap element 520, and the second end cap element 530 is formed of a biodegradable glass, the degradation of which may be used to control the timing of release of the drug from the enclosed reservoir.

In another embodiment, an implantable DDD having a tube element formed from a biodegradable glass may be configured to form openings in the sidewall of the tube element. For example, the tube element may have a plurality of reservoirs sections formed from a single biodegradable glass composition having varying thicknesses at each reservoir. In this embodiment, the device is configured to release the drug from each reservoir at a different time based on the sidewall dissolution characteristics over that reservoir. Alternatively, the portion of the structural element defining each reservoir could be drawn from a different biodegradable glass composition to control the release timing of each reservoir.

In some embodiments, the implantable DDD is modified to provide a single tube element having multiple reservoir sections. In such embodiments, the DDD may include at least one barrier element positioned within an enclosed reservoir and defining a plurality of separate reservoir sections. The DDD further includes a plurality of drug units distributed within one or more of the separate reservoir sections. In one embodiment, the at least one barrier element includes a plurality of barrier elements positioned within the enclosed reservoir and defining a plurality of separate reservoir sections. In one embodiment, the plurality of drug units is distributed such that each of the separate reservoir sections contains one drug unit. In another embodiment, the plurality of drug units is distributed such that one or more of the separate reservoir sections contains multiple drug units. In one embodiment, the at least one barrier element and the plurality of drug units are arranged such that multiple doses of the drug are released sequentially (and in spaced intervals) from the DDD. A more detailed description of the embodiments of multiple-reservoir DDDs is provided below.

In some embodiments, the implantable DDD also includes a coating over at least a portion of the DDD formed from the biodegradable glass. The coating may be configured to control in vivo contact of the biodegradable glass with the biological fluid. For example, in the coating may be configured to absorb light irradiation from a laser source effective to breach the coating and expose the biodegradable glass to the biological fluid. In some embodiments, the coating is in the form of a patterned film having one or more openings configured to control formation of one or more corresponding openings in the biodegradable glass upon exposure to the biological fluid. By controlling the location and size of the erosion or degradation, more repeatable release times may be obtained by limiting the effects of pit corrosion or geometric tolerances of the biodegradable glass. Non-limiting examples of materials suitable for use as coatings in embodiments with the biodegradable glass include silicon nitride, silicon oxide, zinc oxide, titanium nitride, aluminum oxide, titanium oxide, and aluminum nitride.

Prior to loading the drug into the glass tube element, one end of the tube element may be sealed using known techniques for sealing micro-reservoirs. However, alternative methods of sealing an end of the glass tube element may provide benefits in terms of cost, assembly labor, and device performance. Key features of the type of seal used are volume efficiency, hermeticity, biocompatibility, and biostability over the designed lifetime, although the cap may be specifically designed to be degradable in a prescribed time period. In terms of volume efficiency, end cap elements preferably are made as thin as possible with a diameter that is smaller than or equal to the reservoir outer diameter.

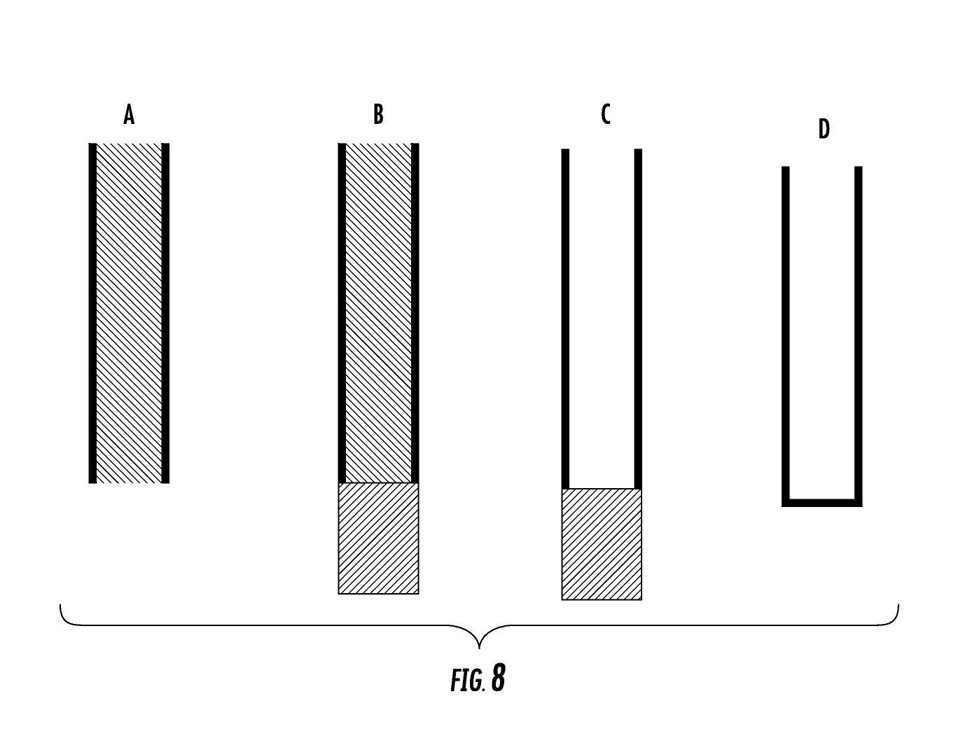

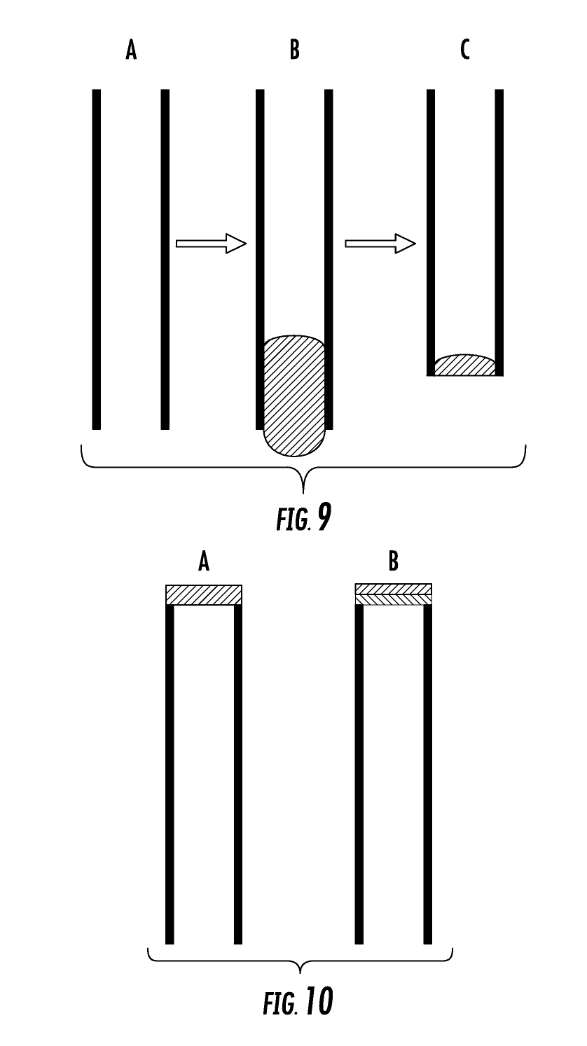

The material and shape of the first and second end cap elements may be varied depending on whether or not the end cap elements function to at least in part control the timing of the release of the drug unit from the enclosed reservoir. FIGS. 6A-D show four examples of end cap construction, in both plan and cross-sectional views. For example, in one embodiment an end cap element is formed from a single hermetic material (FIG. 6A), such as a biodegradable glass that will degrade, dissolve, or hydrolyze when in contact with a biological fluid to compromise the integrity of the end cap element and permit ingress of fluid into the enclosed reservoir and drug diffusion out of the reservoir. In this embodiment, the timing of the drug's release is controlled at least in part by the degradation rate of the material forming the end cap elements, the thickness of the end cap elements, and the mechanism of degradation (surface or bulk) of the end cap elements.

In another embodiment, an end cap element 600 of the implantable DDD may be formed from a biodegradable substrate 602 having one or more thin film coatings 604, 606 (FIG. 6B). The substrate 602 functions largely as a support structure for the thin film coatings 604, 606, degrading rapidly after a biological fluid penetrates the thin film coatings 604, 606. In an embodiment, the substrate 602 is made of a biodegradable glass, biodegradable polymer, or another readily soluble material (e.g., an alkali halide crystal). Non-limiting examples of alkali halide materials include NaCl, KCl, and KBr. In this embodiment, the timing of the drug's release is primarily controlled by the thickness and degradation rate of the material forming the thin film coatings 604, 606. One or more thin film coatings may be used to increase the hermeticity of the device or to tailor the release rate. For example, films with different compositions may be stacked or deposited in alternating layers. Non-limiting examples of materials that may be used in the thin film coatings include silicon nitride, silicon oxide, zinc oxide, titanium nitride, aluminum oxide, and aluminum nitride. In certain embodiments, the films have a thickness from 10 to 5000 nm thick, from 50 nm to 1000 nm, or from 50 nm to 500 nm.

In still another embodiment (FIG. 6C), the end cap elements 610 include a substrate 612 with an aperture 613 in the middle and one or more thin film coatings 614, 616 that fully cover the aperture 613 or that are disposed in the aperture 613. For example, the substrate 612 may be silicon, which may be doped or undoped. In this embodiment, the timing of the drug release is controlled by the degradation rate and thickness of the thin film coatings 614, 616. Two or more thin film coatings may be used to increase the hermeticity of the device, to tailor the release rate, or to increase the mechanical strength of the end cap element. For example, in the embodiment illustrated in FIG. 6C, the thin film coating 614 imparts mechanical strength to the end cap element 610 and is positioned closer to the substrate 612 than the thin film coating 616, which is used to control the timing of release of the drug. Thin film coating 614 degrades/dissolves more quickly than the rate limiting thin film coating 616. The thin film coatings may be made using the same coating materials and methods described above, including silicon nitride, silicon oxide, zinc oxide, titanium nitride, aluminum oxide, and aluminum nitride.

In yet another embodiment (FIG. 6D), the end cap element 620 is a composite material. For example, the end cap element 620 may be formed from a composite having two components: a slower-degrading matrix material 622 and a faster-degrading filler material 624. The filler material 624 preferably is loaded at a sufficiently high concentration to ensure that substantially all filler material particles touch other filler material particles. As the filler material 624 dissolves or degrades, it leaves voids in the matrix material 622 that create pathways for the drug's release. Non-limiting examples of matrix materials include polymers such as acrylates, methacrylates, and biodegradable or non-degradable epoxies. Non-limiting examples of filler materials include nano- or micro-particles made of certain water soluble salts, or metals, such as magnesium or zinc, or nano- or micro-particles made of biodegradable glass, or biodegradable glass flakes.

### 3. Methods of Making Biodegradable Drug Delivery Devices

The tube elements formed of the biodegradable glass may be formed using a drawing process known to those skilled in the art. To form high quality very thin walled glass tubes, there are a number of parameters to consider, including the starting material form and purity, the glass physical properties and thermal properties, and the glass processing history. In terms of the starting material, the glass should be void and defect free and of a uniform composition. The starting material should not contain glass crystallites or other nucleating impurities that will cause the glass to devitrify. Different glass compositions have different propensities for devitrifying in terms of both the devitrification kinetics and temperature. Optimally, the devitrification temperature will be sufficiently different from the glass drawing working temperature or sufficiently slow to allow for a wide processing window. The glass thermal and processing history will influence the final stress state and an annealing step may be necessary to reduce stress. Finally, the glass needs to be relatively physically robust to withstand the drawing process and subsequent handling steps to fabricate final forms. The glass formulation needs to be adjusted so that degradation rates and processing parameters are optimized for the particular application.

In some embodiments, a capped glass capillary may be formed by co-drawing two different glass formulations where one glass, the inner or core glass, is selectively etched without etching the outer or cladding glass. INCOM USA commercially produces glass microwells using this technique on fiber bundle arrays for chemical assays and DNA sequencing applications, although these microwells tend to be very small (on the order of 10 to 100 μm in diameter and a similar depth). For the glass tube elements described herein, diameters on the order of 500 μm are desirable with depths of 500 to 2000 μm.

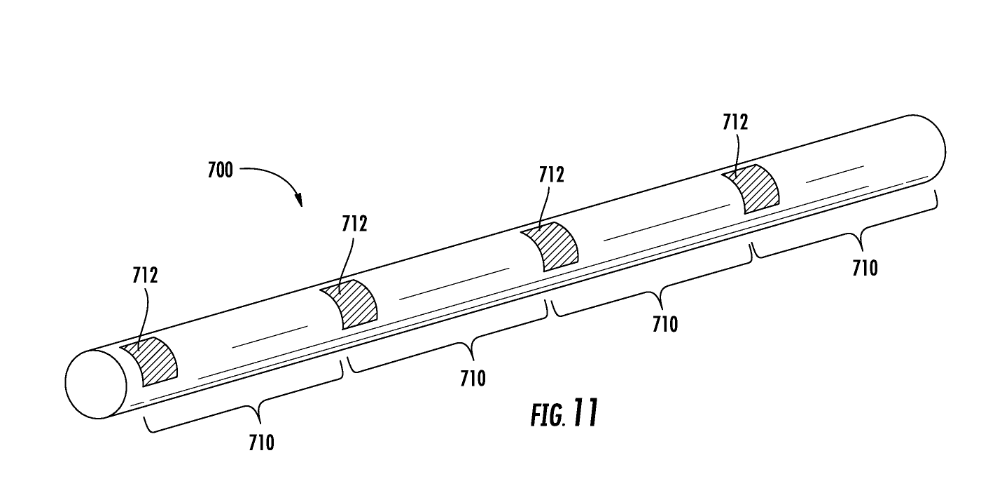

The glass tube elements may be formed by placing a glass tube (cladding) material over a rod (core) material and drawing the materials together. The dimensions of the tube and rod are chosen such that the final draw step produces the diameter and wall thickness of interest on the cladding glass. The core glass and cladding glass are chosen such that an etch selectivity of core to cladding etch rates are 100:1 up to 1000:1 or higher. The glass can then be cleaved and polished into segments that produce the reservoir length of interest (A in FIG. 7). The core glass can then be etched with a selective etchant to produce a glass capillary with a single sealed end (B in FIG. 7). The etch process may occur from a single end by applying an etch protectant to the sealed end (e.g., a photoresist or other suitable polymer). Alternatively, the glass capillary may be etched from both directions leaving a small disk of core material in the center of the capillary followed by a polishing or cleaving step to produce the shape B in FIG. 7. Producing a flat bottom structure may be difficult due to transport issues related to the etchant and by-products. Ideally, the etch rate would be slow relative to the diffusion speeds so that a uniform etch rate will be achieved at both the center and outer diameter of the core material. It may be possible to achieve a uniform etch rate by modifying the chemical constituents of the etchant. It may be necessary to add a convective component to the fluid such as jetting the fluid into the capillary using a microneedle or using ultrasonic energy to aid with mixing. If a uniform etch rate cannot be achieve through chemical or process modifications, it may be possible to add an etch stop. This may be achieved by bonding the structure A shown in FIG. 7 to a rod of the cladding material or another material that is not significantly etched by the core glass etchant as illustrated inFIG. 8. The bonding step may be accomplished by fusion splice or heat sealing the rod material to the co-extruded glass structure (B in FIG. 8). In this embodiment, etching can continue until all of the core material is removed (C in FIG. 8) and then the rod material can be cleaved or polished back to the appropriate thickness (D in FIG. 8).

For tube elements formed of biodegradable glass (FIG. 9), it is possible to form an integral end cap element from the biodegradable glass of the tube element (A in FIG. 9) by a flame seal process. For example, a high temperature heat source (e.g., a flame) can be used to melt the end of the tube element to form a ball-like structure (B in FIG. 9). While this method is simple to implement, it usually is not volume efficient as the ball seal region tends to be thicker than the outer diameter of the tube element. It may be possible, however, to mitigate this effect by polishing back the end seal to a minimum thickness (C in FIG. 9).

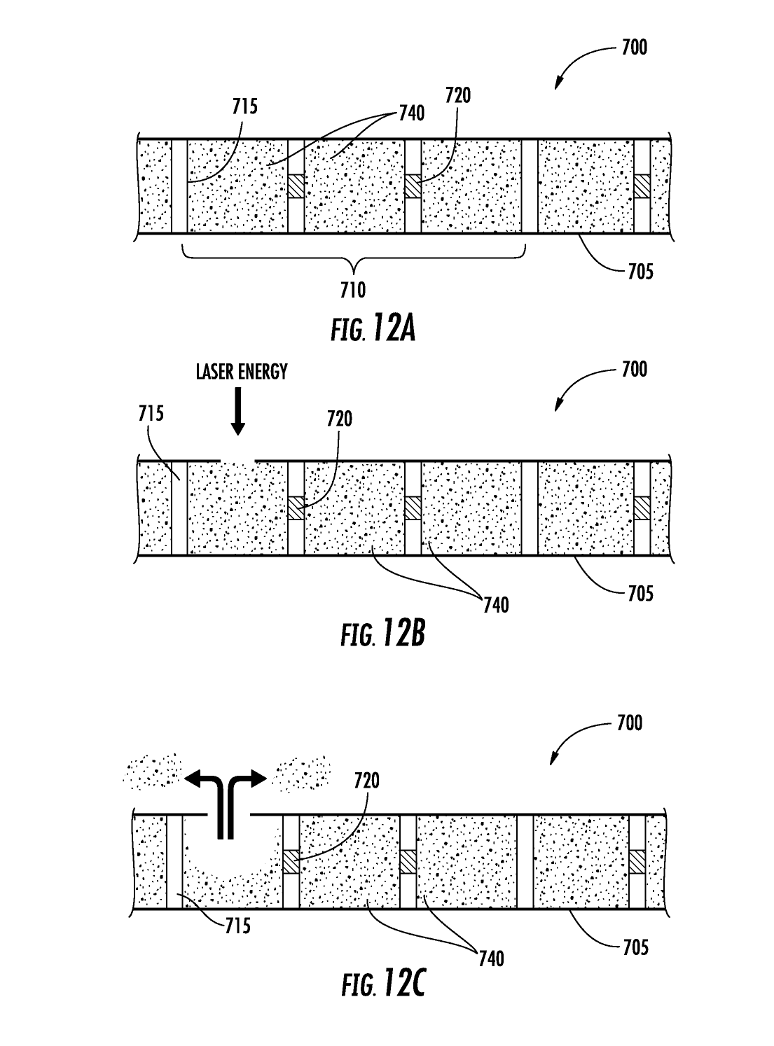

Alternatively, a thin disk of biodegradable glass may be used to form the end cap element cap material by placing the material onto the end of the tube element and heating the end cap element (FIG. 10). In such embodiments, the end cap element may be a single material (A in FIG. 10) or a composite material (B in FIG. 10). For example, the end cap element may be formed from the same biodegradable glass as the tube element, a different formulation of biodegradable glass, a glass frit, or a glass frit layer (preform) in conjunction with a glass end cap element (B in FIG. 10). Heat may be applied using a reflow oven, laser, flame, or any other method known to those skilled in glass forming or bonding. For example, a fiber optic fusion splicer may be used to bond a rod to a glass tube of the same outer diameters followed by a cleaving operation leaving a thin disk of rod material bonded to the tube.

### 4. Multiple Reservoir Drug Delivery Devices

The implantable DDDs described herein may be configured to include a plurality of reservoirs to provide various modes and combinations of drug delivery. As will be apparent from the description and drawings provided herein, the number of reservoirs included in the implantable DDDs may be increased or decreased to achieve a desired release profile. Thus, it is understood 2, 3, 4, 5, or n-dose reservoirs could be constructed as long as other design constraints, such as overall size, are met.

In one embodiment, a plurality of reservoirs is formed within a single tube element using one or more barrier elements. These barrier elements may function to define and temporarily separate adjacent reservoirs from one another. The may be configured to be removable, or more particularly, rupturable or degradable, at a pre-selected time following exposure to an aqueous solution, such as a biological fluid in vivo.

The barrier elements may be formed from a pure material, or can be a laminate, layered, or composite material. Thus, the structure of the barrier elements can be optimized to facilitate the desired drug release profile. For example, the barrier elements may be designed to have similar dissolution rates in order to provide equi-duration dose releases, or may be designed with different durations between doses by changing the geometry of the barrier element or composition of the barrier element.

In some embodiments, the barrier elements are formed from a biodegradable polymer. In other embodiments, the barrier element is a hermetic material that forms a hermetic barrier between adjacent reservoirs. In embodiments, the at least one barrier element includes a plurality of barrier elements including both hermetic materials and biodegradable polymers to create a custom and complex drug release profile.

In one embodiment, the barrier element is formed of a polymer from the class of polyanhydrides or other polymers that are relatively hydrophobic and that degrade over time by a surface erosion mechanism. Accordingly, the barrier element permits release of a drug from a reservoir section after exposure to a fluid for a pre-determined period of time. In one embodiment, the barrier element is formed of a relatively hydrophilic “bulk-eroding” polymer that is highly permeable or soluble. Accordingly, the barrier element permits release of a drug from a reservoir section soon after exposure to a fluid.

In one embodiment, the barrier element is a hermetic material that is configured to dissolve upon exposure to a biological fluid over time. In an embodiment, the hermetic barrier element includes a substrate formed from a non-hermetic, biodegradable material with a coating of a hermetic material. Non-limiting examples of hermetic materials include metals, such as magnesium or iron, thin layers of silicon oxide, silicon nitride, and titanium dioxide, and a bioerodible glass. It should be appreciated, however, that the hermetic barrier between adjacent reservoirs need only provide hermeticity for the pre-determined period of time needed to obtain the desired drug release profile.

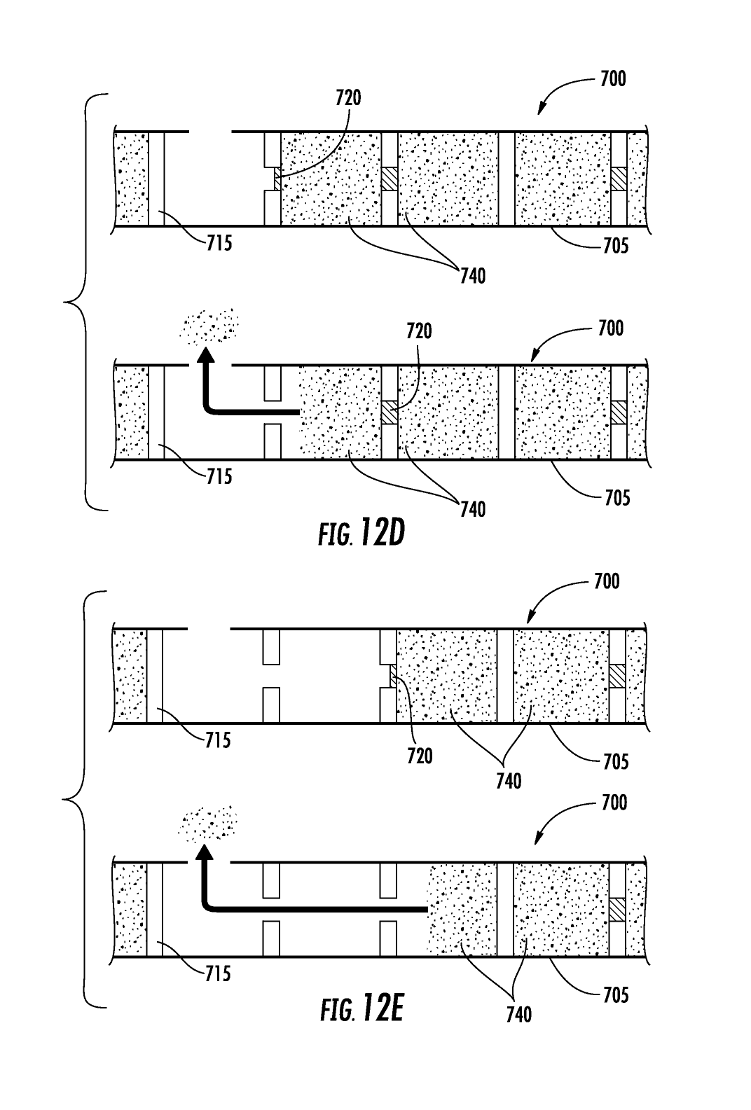

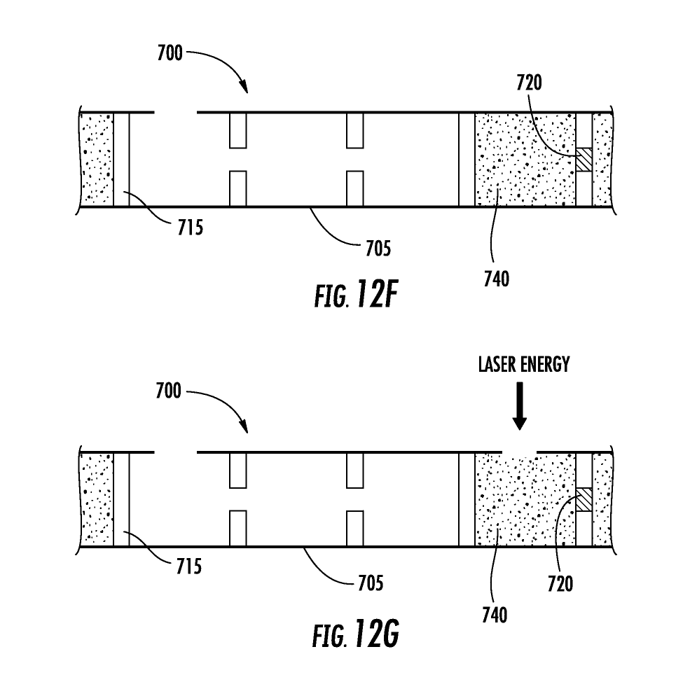

An embodiment of an implantable DDD 700 having several separate reservoir components 710, each having a plurality of reservoirs defined by a plurality of barrier elements 715, is illustrated in FIG. 11 and FIGS. 12A-12G, which provide possible cross-sectional views of its operation by application of an activation stimulus to a plurality of reservoirs joined by hermetic barriers and the subsequent release of the drugs from each of the reservoirs. An activation stimulus (e.g., laser energy) may be applied to a target area 712 on the outer surface of the first reservoir component 710 to rupture the tube element (reservoir) wall 705 and expose the contents 740 of a first reservoir of the first reservoir component 710 (FIG. 12B). The contents 740 of the first reservoir of the first reservoir component 710 are released through the opening (FIG. 12C). During or after release of the contents 740 from the first reservoir, the hermetic barrier 720 is exposed and degrades, ruptures, or otherwise loses its structural integrity, thereby exposing and releasing the contents 740 of the second reservoir of the first reservoir component 710 (FIG. 12D). Similarly, the hermetic barrier 720 between the second and the third reservoirs of the first reservoir component 710 is exposed either during or after release of the contents 740 from the second reservoir of the first reservoir component 710 such that the hermetic barrier 720 degrades, ruptures, or otherwise loses its structural integrity and exposes and releases the contents of the third reservoir of the first reservoir component 710 (FIG. 12E-12F). An activation stimulus can then be applied to another series of reservoirs (i.e., a second or third reservoir component 710), each having a plurality of hermetic barriers 720 to define a plurality of reservoirs, to repeat the process (FIG. 12G). The first, second, and third reservoir component may be separated, for example, by non-removable hermetic barrier 715.

Thus, embodiments of the devices provided herein may be used to provide delivery of drugs in a predetermined sequence and at a predetermined time frame using application of only a single activation stimulus or less frequent application of activation stimulus. For example, in an embodiment the reservoirs may be released in approximately 30 day intervals. In this embodiment, the laser would be used to release a first dose 740 from a first reservoir component 710 (e.g., FIG. 12B-12C). After that first reservoir is emptied and the hermetic barrier 720 material is exposed and degraded or otherwise removed, the second dose 740 would be released through the opening created in the first reservoir around day 30 (e.g., FIG. 12D). Then, when the second reservoir has been emptied and the hermetic barrier 720 material is exposed and degraded or otherwise removed, the third dose 740 would be released through the opening created in the first reservoir around day 60. At day 90, an activation stimulus would be applied to a target area 712 of the second reservoir component 710 in the DDD, and the process would repeat itself.

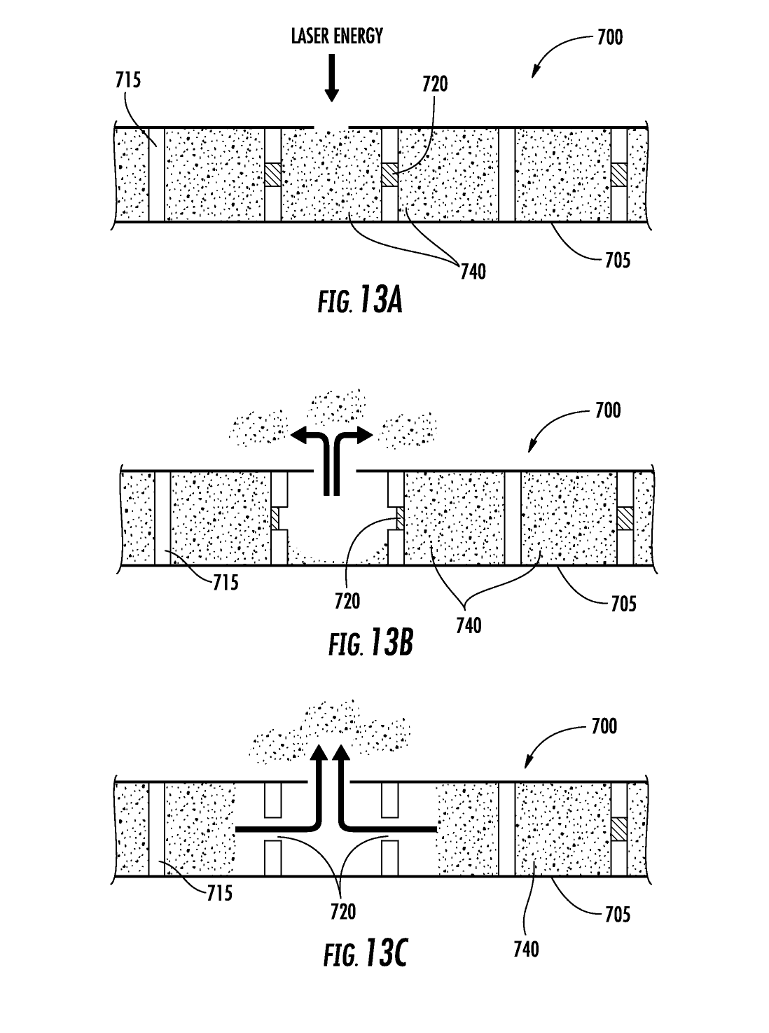

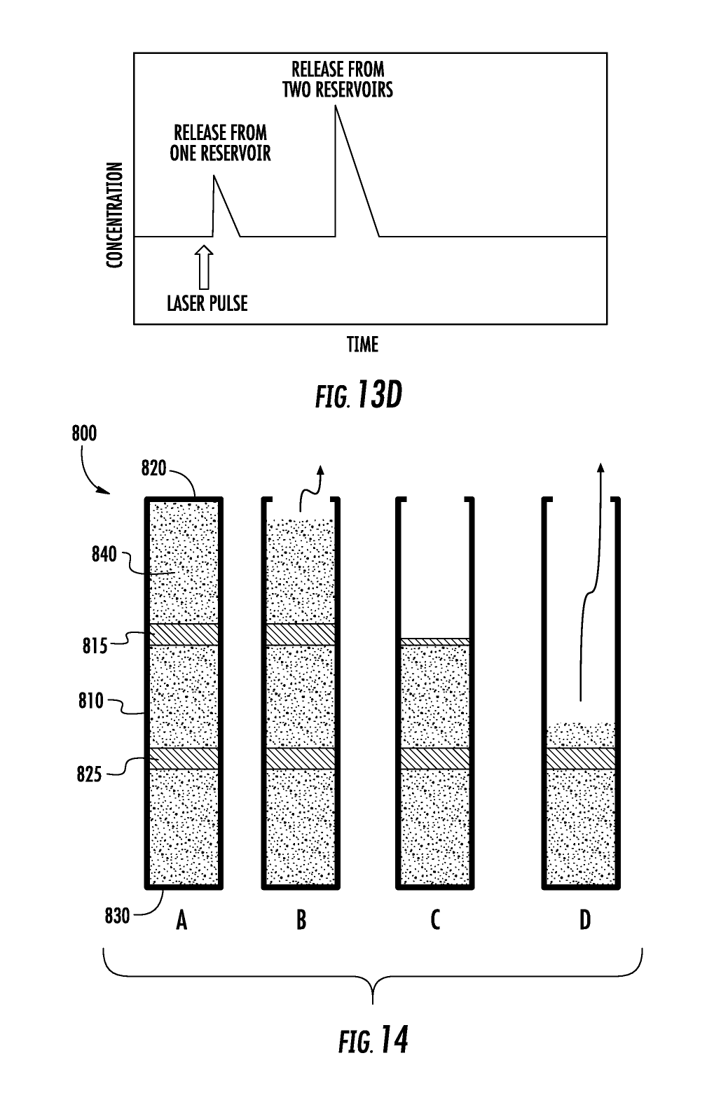

An implantable DDD having a plurality of reservoirs defined by hermetic barriers 720 also is described by reference to FIGS. 13A-13C, which provide another possible cross-sectional view of the operation of the device of FIG. 11. For example, an activation stimulus may be applied to a target area on a middle reservoir to expose the contents of the middle reservoir (FIG. 13A). After the contents 740 of the middle reservoir are released, and the hermetic barriers 720 on the two adjacent reservoirs are exposed (FIG. 13B), the contents 740 of the two adjacent reservoirs may be released simultaneously to increase the amount of drug released (FIG. 13C-13D). Alternatively, the hermetic barrier on a first of the adjacent reservoirs may be thicker than the hermetic barrier on a second of adjacent reservoirs, such that the dose of the first adjacent reservoir is released prior to the dose of the second adjacent reservoir (not shown). In another embodiment, a shielding element capable of absorbing light irradiation may be disposed in the middle reservoir instead of a drug unit, thereby preventing the laser heat and radiation from compromising the drug. In this situation, the middle reservoir functions as a protective release mechanism. For example, the middle reservoir may be filled with a shielding element in the form of a thin titanium rod dimensioned to absorb the laser energy while permitting fluid to move between the rod and hermetic barriers to erode the barriers and provide for the release of the drug.

In another embodiment, an implantable DDD having a plurality of separate reservoir sections is configured to release drug passively for an extended period without the need for application of an activation stimulus. The operation of one embodiment of such a device is illustrated, for example, in FIG. 14. The DDD 800 includes a tube element 810 with first 820 and second 830 end cap elements. Two removable barrier elements 815, 825 are disposed in the enclosed reservoir of the tube element 810 to define formed three reservoir sections, each having a drug unit 840. While a three-reservoir DDD is illustrated, it is understood that 2-, 4-, or 5-, or n-reservoir DDDs could be similarly constructed. In FIG. 14, A shows, the end cap element 820, which is formed from a biodegradable material, is shown as providing a thinner barrier than the two removable barrier elements 815, 825. This permits for a rapid release from the DDD 800 immediately after implantation while still maintaining the hermeticity of the DDD 800 prior to implantation. The rapid release can be controlled by changing the geometry of the end cap element (e.g., thickness), by changing the material composition (e.g., glass formulation), or by combinations thereof. After the end cap element 820 has dissolved or otherwise been compromised, the first drug 840 is released from the first reservoir section (B in FIG. 14) and the first removable barrier element 815 starts to dissolve (C in FIG. 14). After the first removable barrier element 815 dissolves or is otherwise compromised, the second drug 840 is released from the second reservoir section and the process continues (D in FIG. 14).

In some embodiments, the multiple reservoirs are provided by combining two or more separate reservoir components in an implantable DDD. The reservoir components may be combined in an implantable DDD in any of a number of different ways. For example, an implantable DDD may include a plurality of separate reservoir components that are connected by an external structural element. Non-limiting examples of external structural elements that may be used to secure the separate reservoir components together include a degradable or non-degradable epoxy or other adhesive; a backing made of a degradable or non-degradable material, including but not limited to a degradable polyester (e.g., poly(lactic-co-glycolic acid)) or a non-degradable polyester (e.g., poly(ethylene terephthalate)); a degradable or non-degradable suture material; a degradable or non-degradable glass fiber; a perforated or woven metal or polymer tube; a coating (e.g., parylene); or a flexible tether. Although the external structural element primarily functions to hold the reservoir components together (typically in an axial, or end-to-end arrangement), the external structural element also may be configured to have the same function as the coatings described herein (i.e., providing the desired hermetic properties or light irradiation absorbing properties).

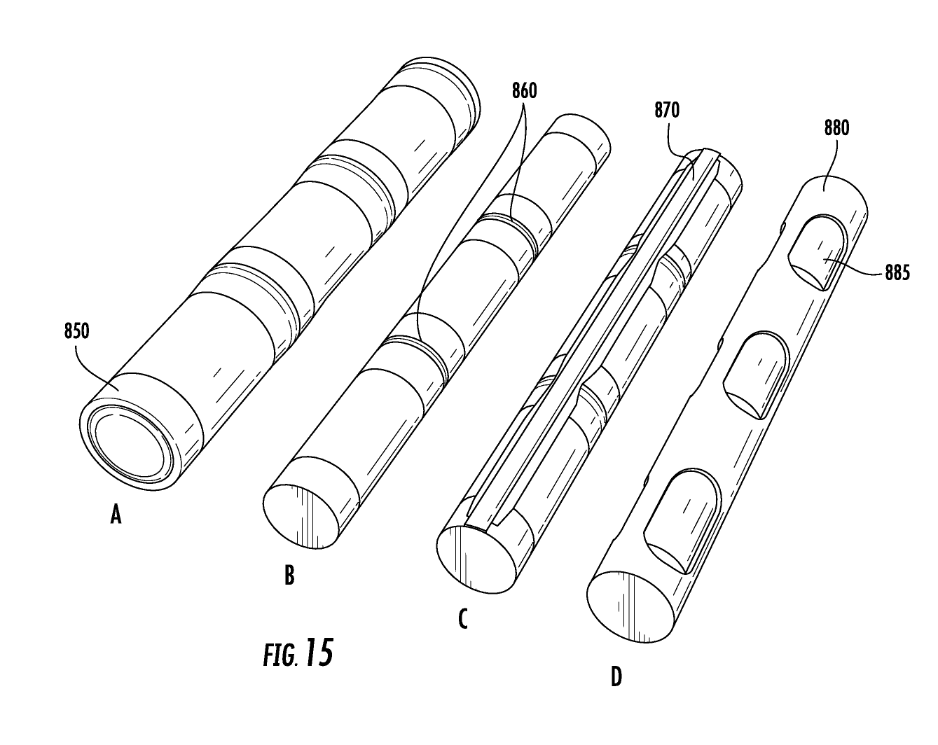

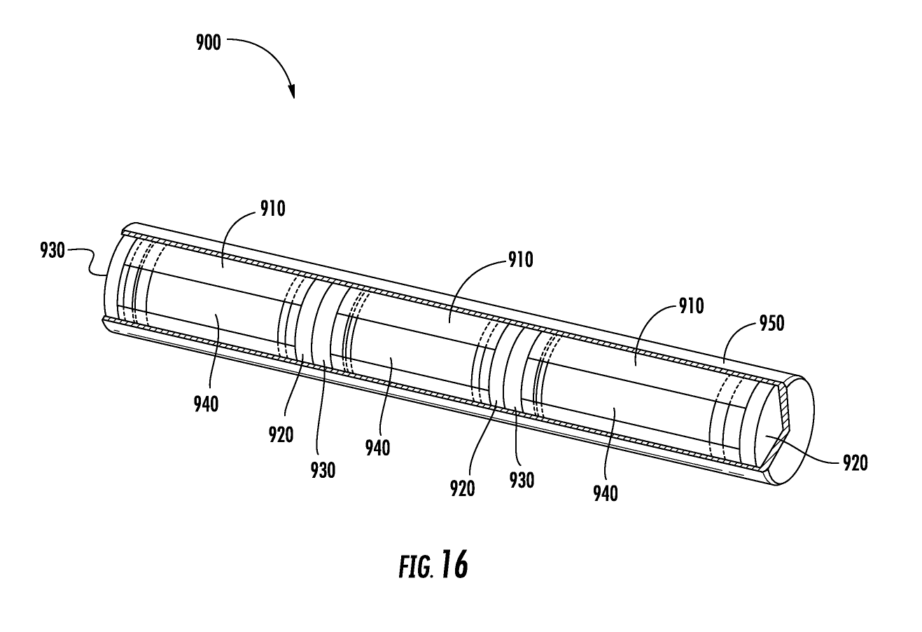

Embodiments of external structural elements used to connect the separate reservoir components are illustrated in FIG. 15A to 15D. In FIG. 15A, the implantable DDD includes three separate reservoir components aligned end-to-end within a polymer tube. In FIG. 15B, three separate reservoir components are connected end-to-end using a suitable adhesive disposed between the reservoir components. Alternatively, in FIG. 15C, three separate reservoir components are connected end-to-end using one or more strips of material applied to the outer surface of the reservoir components. This strip may be formed from any material that is biocompatible and having sufficient structural integrity to maintain the assembly over prolonged periods in vivo, non-limiting examples of which include polymers such as PET, metals such as titanium, or ceramics such as alumina. In this configuration, the reservoir components may be adhered to the strip using a suitable adhesive, such as a biocompatible epoxy or silicone. Another exemplary DDD includes a micromachined or cast sheath material encapsulating the separate reservoir components (FIG. 15D). The material optionally may include one or more openings for application of the laser energy. Non-limiting examples of materials that may be used to form the sheath include polymers, such as PET or silicone, and metals, such as titanium.

An exemplary embodiment of an implantable DDD having a plurality of separate reservoir components is illustrated in FIG. 16. The three separate reservoir components 910 are aligned end-to-end in the implantable DDD 900. Although three reservoir components are shown, any suitable numbers of separate reservoir components may be incorporated into a DDD. An external structural element 950 is a polymer tube formed surrounding the three separate reservoir components. In this DDD, each separate reservoir component releases the drug 940 from the enclosed reservoir(s) independently, and the timing may be controlled by degradation of the tube element 910 defining the separate reservoir components, degradation of one or both end cap elements 920, 930, and/or absorption of light irradiation effective to open the enclosed reservoir(s) to permit release of the drug of drug unit 940.

For example, in one embodiment the end cap elements of each reservoir are degradable such that the fluid moves into the reservoir at a proximal end of the DDD (i.e., after the first end cap element is sufficiently degrades to permit fluid ingress), and through the DDD toward the reservoir at a distal end of the DDD, as the end cap elements of each separate reservoir component degrade and the drug is released. The drug release is sequential, starting from the first reservoir at the proximal end and repeating periodically as the next reservoir opens. Desirably, biological fluid does not penetrate the space between the tube element and the structural element such that the structural element slows or prevents water ingress into the device from all pathways except the open end. Thus, the timing of the release of the first drug is controlled by degradation/dissolution of the first end cap element exposed to the biological fluid. Subsequent drug release is controlled by the degradation of two end cap elements: the second end cap element of the open reservoir, and the first end cap element of the next sealed reservoir. These end cap elements may be made of the same or different degradable materials, and may be designed to degrade at the same rate or different rates. In a preferred embodiment, DDD elements are formed from biodegradable materials, forming a fully biodegradable device. Thus, to prevent premature drug release, the external support structure and/or tube element must degrade sufficiently slowly to prevent any undesired ingress of the biological fluid into DDD until after all doses of drug are released.

The DDD may further include separate reservoir components separated by degradable barriers. That is, the DDD includes alternating reservoir components and degradable barriers configured to completely separate each adjacent reservoir component. Each reservoir component is formed of a tube element. The tube element may be formed from a biodegradable glass or a non-degradable glass or metal (e.g., titanium). In embodiments in which the tube element is degradable, the tube element should be configured to degrade after the drug is released. The barriers can be made from any of the same designs and materials as the end cap elements described above. The initial drug release begins with degradation of the first end cap element at one end of the DDD, and subsequent doses of the drug are released as each barrier degrades sequentially. All barriers may be identical, resulting in pulsatile release at uniform intervals, or the barriers may be different, resulting in a burst release at desired time points. The final end cap element may be degradable or non-degradable. In embodiments in which the final end cap element is degradable, the end cap element should be configured to degrade after the final dose of drug is released. Alternately, both end cap elements may degrade on a similar time scale, allowing drug release to occur from both ends of the device. In this case, drug release continues to occur sequentially from both ends as subsequent barriers erode, and the order and timing of release from each reservoir depends on the degradation rate of each barrier that comes into contact with biological fluid. To prevent premature degradation of the barriers from contact with the biological fluid, a hermetic coating or sealant may be applied at least at the joints of the separate reservoir components and barriers.

In another embodiment (not shown) the DDD may also be attached to an external structural element that may be used, for example, to attach additional components (e.g., suture loops), to protect the DDD from damage during handling or insertion, or to increase the stiffness of the DDD.

Advantageously, the implantable DDDs provided herein are capable of controlling the storage environment of the drug in the reservoir until the time selected for its release. Specifically, the implantable DDDs provided herein may be hermetically sealed to exclude water ingress into the reservoir of the implantable DDD in order to maintain the stability of the drug for prolonged periods (i.e., of months, a year, a more), such as during storage of the device before implantation and following implantation in vivo until after each reservoir is selectively activated (e.g., ruptured to release the drug contained therein).

### 5. Laser Activation

The laser-activated DDDs described herein facilitate non-invasive release of a drug in a tissue being treated. In embodiments, the DDDs may be used to facilitate release of a drug into an ocular tissue, such as the macula or retina. For example, the DDDs may permit release of the drug into the posterior chamber through the vitreous portion of the eye or through the conjunctiva, sclera, or choroid. In addition to being non-invasive, laser activation allows for multiple dosing from a single DDD having multiple reservoir sections or multiple reservoir components.

The DDDs permit release of a drug payload when triggered by a pulse of light irradiation. In some embodiments, the light irradiation is a focused laser. In some embodiments, as described above, the DDD includes a structural element (e.g., a tube element and/or an end cap element) and/or a coating formed of an irradiation-absorbing material. Additionally, the structural element and/or coating is able to absorb the pulse of light irradiation, which heats the structural element and/or coating as well as adjacent elements of the DDD. In some embodiments, the structural element and/or coating is formed of an irradiation-absorbing material having a high optical absorption coefficient at a wavelength appropriate to a laser device to be controlled by a user for release of the chemical substance. In some embodiments, as described above, the structural element and/or coating has a thickness that is substantially thinner and more mechanically fragile than other elements of the DDD. Accordingly, a breach may be formed in the structural element and/or coating upon sufficient heating from the light irradiation. For example, the structural element and/or coating may fracture or melt near the area where the light irradiation is applied. In some embodiments, the heating of the structural element and/or coating is sufficient to form a breach in an adjacent element surrounding the enclosed reservoir.

In some embodiments, the implantable laser-activated DDDs described herein (e.g., FIG. 1) include a tube element having a thin wall thickness and formed of a material that is able to absorb the light irradiation. Accordingly, the tube element may fracture upon application of the light irradiation, opening the enclosed reservoir to permit release of the drug. In some embodiments, the DDD includes a thin coating formed over all or a portion of the tube element and thermally coupled to the tube element. The coating may be formed of an irradiation-absorbing material, and the tube element may be formed of a material that does not absorb light irradiation. Accordingly, the coating and the tube element may fracture upon application of the light irradiation, opening the enclosed reservoir to permit release of the drug.

### 6. Methods of Using the Drug Delivery Devices

The implantable DDDs described herein may be used to deliver a drug to a patient. Particularly, the implantable devices facilitate selective release of a drug to the interior of the eye for the treatment of ocular conditions. However, the implantable devices may be adapted for use in other parts of the body.

One embodiment includes a method of delivering a drug to a patient by use of a laser-activated DDD. The method includes implanting one of the above-described DDDs into a tissue site of the patient. The device includes a drug contained in an enclosed reservoir. The method also may include irradiating at least a portion of the DDD to breach the enclosed reservoir to permit the drug to be released in tissues at the tissue site. In some embodiments, the DDD may be configured to contain and release multiple doses of one drug or combinations of drugs. Different drugs may be disposed in separate reservoirs or mixtures of two or more drugs may be disposed in each reservoir.

In some embodiments, the tissue site is ocular tissue. In one embodiment, the tissue site is in the posterior chamber of the eye. In one embodiment, the tissue site is in, on, or under the conjunctiva of the eye. In one embodiment, the tissue site is in, on, or under the sclera of the eye. In one embodiment, the tissue site is in, on, or under the choroid of the eye.

In some embodiments, the tissue site is the brain tissue. Embodiments of the hermetically sealed devices described herein that do not require the input of energy to open, such as those using degradable or hermetic materials/barriers, can be inserted in the brain, and drug release therefrom would be controlled, at least in part, by the choice of degradable materials and the structure of the device. Such devices would be capable of providing drug to the brain in a way similar to the Gliadel® wafer and other conventional polymer depots—but, unlike those depot systems, the DDDs described herein provide hermeticity, thereby beneficially enabling the storage and delivery of drug molecules that are sensitive to humidity and/or otherwise would have limited stability in a non-hermetic system.

In an embodiment, if it is desired to provide precise control of the timing of release from DDD for the brain, the DDD described herein may be configured to take advantage of systems designed for targeting energy to precise locations in the brain. For example, Stereotactic Radiosurgery (SRS) is a method of delivering high dose radiation to a precise location in a body tissue. Two examples of systems used to deliver such targeted radiation to body tissues are the Gamma Knife® by Elekta and the CyberKnife®. The Gamma Knife® is generally designed only for use in the brain, and the CyberKnife® can be used in the brain and other locations in the body. They differ in how they deliver (e.g., source of radiation, method of beam focus, etc.) the radiation beams to the patient, but they are similar in that they can precisely control the location of the radiation to less than 1 mm. Such precision in energy delivery may allow these systems to deliver energy to a single drug-filled reservoir of an implanted, hermetically sealed multi-reservoir device, as described herein, that has been implanted in or adjacent to the brain.

In an embodiment, a CT scan, MRI, or X-ray may be used to locate the device in the brain, and the system may be used to target the radiation to a particular location on the DDD. The energy may disrupt the outer barrier of the reservoir and allow the drug to be released from the DDD. The energy dose, the time of application, and the target location on the DDD may be selected based on the geometry of the implant, its materials of construction, and the mechanism by which the energy disrupts the outer barrier. This mechanism could be localized heating and melting like that from certain lasers or could be some other type of radiation-induced damage or change in the mechanical properties of the material. Such embodiments also may include a shielding element in the enclosed reservoir to protect the drug.

In one embodiment, the DDD is configured for use in the treatment of cancer. In one particular embodiment, the drug includes temozolomide (TMZ), known commercially as Temodar®, Temodal®, or Temcad®. DDD for treatment of cancer may also include another drug, such as O6-benzylguanine (O6BG), which has been shown to increase the efficacy of TMZ if O6BG is delivered to the brain cancer cells before the TMZ. Therefore, in one embodiment, the DDD includes a plurality of reservoirs with the drugs disposed in different reservoirs. In use, the O6BG reservoirs are opened first with targeted radiation to pre-treat the cancer cells, and then a suitable time later, the TMZ reservoirs are opened with a second targeted of radiation. By placing DDDs with varying payloads in several locations around a tumor in the brain, the physician has excellent control over what drug is delivered at what time and at what location, and can treat the tumor based on how it progresses without having to go back into the brain surgically. In other words, the delivery of the drugs from the DDDs is non-invasive because it uses these non-invasive SRS techniques to control the implanted DDD.

Other diseases of the brain that could be treated in this way include Parkinson's disease, Huntington's disease, and Alzheimer's disease, among others.

The DDDs described herein also may be used for treatment of tissue sites in other locations of the body. Non-limiting examples of other tissue sites include the spine, joints, liver, bladder, lungs, heart, etc., and SRS technologies like the CyberKnife® may be used to release drug from those devices.

To the extent that descriptions, definitions, and terms in material that is incorporated by reference conflict with descriptions, definitions, and terms expressly included in this specification, the description, definition, and terms expressly included in this specification should govern.

While the present invention has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereof.

We claim:

1. A drug delivery device comprising:
a tube element having a reservoir enclosed therein, the tube element having a first open end and an opposing second open end, the first and second open ends being closed off, respectively, by a first end cap element and a second end cap element, such that the tube element, the first end cap element, and the second end cap element together define the enclosed reservoir;
a drug unit contained in the enclosed reservoir, the drug unit comprising a drug; and
a shielding element contained in the enclosed reservoir,
wherein the drug delivery device is configured to absorb light irradiation from a laser source effective to rupture the tube element, thereby opening the enclosed reservoir to permit release of the drug from the drug delivery device, and the shielding element being configured to shield the drug unit from the light irradiation.

2. The drug delivery device of claim 1, wherein the first end cap element is joined to the first open end of the tube element at a first joint and the second end cap element is joined to the second open end of the tube element at a second joint, wherein the first and second joints are hermetically sealed.

3. The drug delivery device of claim 2, wherein a coating is disposed over the first and second joints.

4. The drug delivery device of claim 1, wherein the shielding element is positioned adjacent to the drug unit in the enclosed reservoir to define a portion of the enclosed reservoir that is devoid of the drug unit.

5. The drug delivery device of claim 4, wherein the shielding element fills less than all of the enclosed reservoir portion that is devoid of the drug unit.

6. The drug delivery device of claim 4, wherein the drug delivery device is configured to absorb light irradiation at a portion of the tube element that is adjacent to the enclosed reservoir portion that is devoid of the drug unit.

7. The drug delivery device of claim 1, wherein the shielding element comprises a cylindrical structure disposed between at least a portion of the drug unit and an inner wall of the tube element.

8. The drug delivery device of claim 7, wherein the cylindrical structure is selected from the group consisting of a cylindrical band, a cylindrical coil, and a perforated cylinder.

9. The drug delivery device of claim 1, wherein the tube element is formed of a metal or a glass.

10. The drug delivery device of claim 1, further comprising a biodegradable barrier element dividing the enclosed reservoir into a first reservoir section containing the drug unit and a second reservoir section which contains a second drug unit.

11. The drug delivery device of claim 10, wherein the biodegradable barrier element comprises a biodegradable glass.

12. The drug delivery device of claim 10, wherein biodegradable barrier element provides a hermetic seal between the first and second reservoir sections.

13. A drug delivery device comprising:
a tube element having a reservoir enclosed therein;
a drug unit contained in the enclosed reservoir, the drug unit comprising a drug; and
wherein the drug delivery device is configured to absorb light irradiation from a laser source effective to rupture the tube element, thereby opening the enclosed reservoir to permit release of the drug from the drug delivery device,
wherein the drug unit is shaped and dimensioned to reside in the enclosed reservoir at a position which creates a buffer zone between a portion of an inner wall of the tube element and the drug unit, the drug unit having a cone-shaped end facing the buffer zone, whereby the buffer zone reduces or eliminates exposure of the drug unit to the light irradiation or heat therefrom.

14. The drug delivery device of claim 13, wherein the drug delivery device is configured to absorb light irradiation at a portion of the tube element that is adjacent to the buffer zone.

15. The drug delivery device of claim 13, further comprising a shielding element configured to shield the drug unit from the light irradiation.

16. The drug delivery device of claim 13, wherein the tube element is formed of a metal or a glass.

17. The drug delivery device of claim 13, further comprising a coating over at least a portion of an outer surface of the drug delivery device.

18. The drug delivery device of claim 13, further comprising a biodegradable barrier element dividing the enclosed reservoir into a first reservoir section containing the drug unit and a second reservoir section which contains a second drug unit.

19. The drug delivery device of claim 18, wherein the biodegradable barrier element comprises a biodegradable glass.

20. The drug delivery device of claim 19, wherein biodegradable barrier element provides a hermetic seal between the first and second reservoir sections.

21. A drug delivery device comprising:
a tube element having a reservoir enclosed therein, the tube element having a first open end and an opposing second open end, the first and second open ends being closed off, respectively, by a first end cap element and a second end cap element, such that the tube element, the first end cap element, and the second end cap element together define the enclosed reservoir;
a drug unit contained in the enclosed reservoir, the drug unit comprising a drug; and
wherein the drug delivery device is configured to absorb light irradiation from a laser source effective to rupture the tube element, thereby opening the enclosed reservoir to permit release of the drug from the drug delivery device,
wherein the drug unit is shaped and dimensioned to reside in the enclosed reservoir at a position which creates a buffer zone between a portion of an inner wall of the tube element and the drug unit, whereby the buffer zone reduces or eliminates exposure of the drug unit to the light irradiation or heat therefrom.

22. The drug delivery device of claim 21, wherein the first end cap element is joined to the first open end of the tube element at a first joint and the second end cap element is joined to the second open end of the tube element at a second joint, wherein the first and second joints are hermetically sealed.

23. The drug delivery device of claim 21, further comprising a biodegradable barrier element dividing the enclosed reservoir into a first reservoir section containing the drug unit and a second reservoir section which contains a second drug unit, wherein the biodegradable barrier element provides a hermetic seal between the first and second reservoir sections.

24. The drug delivery device of claim 23, wherein the biodegradable barrier element comprises a biodegradable glass.
